(19) United States
(12) Patent Application Publication
(10) Pub. No.: US 2009/0286299 A1
(43) Pub. Date: Nov. 19, 2009

(54) ENGINEERED LUCIFERASES

(76) Inventors: Mostafa Ronaghi, San Diego, CA (US); Helmy A. Eltoukhy, Woodside, CA (US); Leila Bazargan, Palo Alto, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/467,222

(22) Filed: May 15, 2009

Related U.S. Application Data
(60) Provisional application No. 61/053,649, filed on May 15, 2008.

(51) Int. Cl. C12N 9/02; C12N 11/00

(52) U.S. Cl. 435/174; 435/189

(57) ABSTRACT

DNA sequencing techniques are important for a variety of research and diagnostic applications. Pyrosequencing is a “sequencing by synthesis” technique that makes use of luciferase. Modified luciferase enzymes and methods of DNA pyrosequencing are provided. Means of preparing and producing mutant luciferases that have enhanced selectivity for ATP or dATP are described.

### RELATED APPLICATIONS

This application claims priority to U.S. Provisional Application No. 61/053,649 entitled “Engineered Luciferases” filed on May 15, 2008, which is incorporated herein by reference in its entirety.

### REFERENCE TO SEQUENCE LISTING

The present application is being filed along with a Sequence Listing in electronic format. The Sequence Listing is provided as a file entitled ILLINC129ASEQLIST.TXT, created May 15, 2009, which is 60 Kb in size. The information in the electronic format of the Sequence Listing is incorporated herein by reference in its entirety.

### FIELD OF THE INVENTION

The present invention relates to the fields of chemistry and biology. In particular, embodiments of the present invention relate to the engineered luciferase enzymes and application of such enzymes in nucleic acid sequencing.

### BACKGROUND

Luciferase enzymes are known from a variety of bioluminescent species. Such enzymes are capable of oxidizing a substrate (for example, luciferin or luciferyl adenylate) with an oxidizing agent (for example, molecular oxygen), thereby producing light. Luciferase enzymes have been employed in a wide variety of applications. One common use of luciferase is as a reporter enzyme in genetic expression studies. More recently, luciferase has been used for signal generation in polymer sequencing applications.

### SUMMARY

Some embodiments of the present invention relate to modified luciferases having a selectivity for ATP over dATP that is greater than the selectivity for ATP over dATP of an unmodified luciferase. In some embodiments of the present invention, nucleic acids encoding modified luciferases, methods for making modified luciferases, methods for obtaining sequence information, and apparatuses for obtaining sequence information comprising modified luciferases are described.

Some embodiments of the present invention relate to modified luciferases. In some such embodiments, a modified luciferase comprises a selectivity for ATP over dATP that is at least three-fold greater than the selectivity for ATP over dATP of an unmodified luciferase from the same organism. In some embodiments of the above-described luciferases, the selectivity for ATP over dATP is at least eight-fold greater then the selectivity for ATP over dATP of an unmodified luciferase from the same organism. In preferred embodiments, the selectivity for ATP over dATP is at least ten-fold greater then the selectivity for ATP over dATP of an unmodified luciferase from the same organism. In more preferred embodiments, the selectivity for ATP over dATP is at least fifteen-fold greater then the selectivity for ATP over dATP of an unmodified luciferase from the same organism.

In some embodiments, the above-described luciferases are modified to further comprise a light emitting activity that is greater than the light emitting activity of said unmodified luciferase.

In some embodiments of the above-described luciferases, a modified luciferase can have an altered amino acid sequence as compared to the amino acid sequence of an unmodified luciferase from the same organism. In some such embodiments, the modified luciferase is from Photinus pyralis. In some embodiments, the altered amino acid sequence comprises an amino acid substitution. In some embodiments, the amino acid substitution is located in the primary amino acid sequence between the N-terminus and the first amino acid forming the active site of said modified luciferase. In other embodiments, the amino acid substitution comprises a conservative amino acid substitution. In still other embodiments, the amino acid substitution is selected from the group consisting of N197, S198, H244, 1423 and any combination thereof. In some such embodiments, the amino acid substitution is selected from the group consisting of N197F, S198T, H244F, I423Y and any combination thereof.

In some embodiments of the above-described luciferases, a modified luciferase can further comprise greater thermostability than said unmodified luciferase.

In some embodiments of the above-described luciferases in which the altered amino acid sequence comprises an amino acid substitution, a modified luciferase can further comprise greater thermostability than said unmodified luciferase. In some such embodiments, the amino acid substitution is selected from the group consisting of T214, I232, F295, E354, and any combination thereof. In certain preferred embodiments, the amino acid substitution is selected from the group consisting of T214A, 1232A, F295L, E354K, and any combination thereof.

In some embodiments of the above-described luciferases, a modified luciferase can further comprise a biotin binding moiety. In some embodiments of the above-described luciferases in which the altered amino acid sequence comprises an amino acid substitution, a modified luciferase can further comprise a biotin binding moiety. In some such embodiments, the biotin binding moiety comprises a polypeptide. In certain embodiments, the polypeptide has at least 70% identity to SEQ ID NO: 3. In some embodiments, the amino acid substitution is selected from the group consisting of T214A, I232A, F295L, E354K, N197F, S198T, H244F, I423Y and any combination thereof.

In some embodiments of the above-described luciferases an amino acid substitution, the amino acid substitution within 10 Angstroms of a binding site for a nucleotide or a binding site for luciferin in the tertiary structure of the modified luciferase. In some such embodiments, the amino acid substitution is within 5 Angstroms of the binding site for a nucleotide or the binding site for luciferin in the tertiary structure of the modified luciferase.

In some embodiments of the above-described luciferases, a modified luciferase can be associated with a solid support. In some such embodiments, the solid support comprises a particle.

More embodiments of the present invention can include nucleic acids encoding a modified luciferase in which the modified luciferase comprises a selectivity for ATP over dATP that is at least three-fold greater than the selectivity for ATP over dATP of an unmodified luciferase from the same organism. In some such embodiments, the selectivity for ATP over dATP is at least eight-fold greater then the selectivity for ATP over dATP of an unmodified luciferase from the same organism. In preferred embodiments, the selectivity for ATP over dATP is at least ten-fold greater then the selectivity for ATP over dATP of an unmodified luciferase from the same organism. In more preferred embodiments, the selectivity for ATP over dATP is at least fifteen-fold greater then the selectivity for ATP over dATP of an unmodified luciferase from the same organism.

In some embodiments of the above-described nucleic acids, the encoded modified luciferase further comprises a light emitting activity that is greater than the light emitting activity of said unmodified luciferase.

In other embodiments of the above-described nucleic acids, the encoded modified luciferase comprises an altered amino acid sequence as compared to the amino acid sequence of an unmodified luciferase from the same organism. In some such embodiments, the encoded modified luciferase is from Photinus pyralis. In some embodiments, the altered amino acid sequence comprises an amino acid substitution. In some embodiments, the amino acid substitution is located in the primary amino acid sequence between the N-terminus and the first amino acid forming the active site of said modified luciferase. In other embodiments, the amino acid substitution comprises a conservative amino acid substitution. In still other embodiments, the amino acid substitution is selected from the group consisting of N197, S198, H244, 1423 and any combination thereof. In some such embodiments, the amino acid substitution is selected from the group consisting of N197F, S198T, H244F, I423Y and any combination thereof.

In some embodiments of the above-described nucleic acids, the encoded modified luciferase further comprises greater thermostability than the unmodified luciferase.

In some embodiments of the above-described nucleic acids in which the altered amino acid sequence comprises an amino acid substitution, the encoded modified luciferase further comprises greater thermostability than said unmodified luciferase. In some such embodiments, the amino acid substitution is selected from the group consisting of T214, I232, F295, E354, and any combination thereof. In certain preferred embodiments, the amino acid substitution is selected from the group consisting of T214A, I232A, F295L, E354K, and any combination thereof.

In some embodiments of the above-described nucleic acids, the encoded modified luciferase further comprises a biotin binding moiety. In some embodiments of the above-described nucleic acids in which the altered amino acid sequence comprises an amino acid substitution, the encoded modified luciferase further comprises a biotin binding moiety. In some such embodiments, the biotin binding moiety comprises a polypeptide. In certain embodiments, the polypeptide has at least 70% identity to SEQ ID NO: 3. In some embodiments, the amino acid substitution is selected from the group consisting of T214A, I232A, F295L, E354K, N197F, S198T, H244F, I423Y and any combination thereof.

In some embodiments of the above-described nucleic acids encoding a modified luciferase having an amino acid substitution, the amino acid substitution within 10 Angstroms of a binding site for a nucleotide or a binding site for luciferin in the tertiary structure of the modified luciferase. In some such embodiments, the amino acid substitution is within 5 Angstroms of the binding site for a nucleotide or the binding site for luciferin in the tertiary structure of the modified luciferase.

In some embodiments of the above-described nucleic acids, the encoded modified luciferase is attached to a solid support. In some such embodiments, the solid support comprises a particle.

More embodiments of the present invention include vectors comprising a nucleic acid encoding a modified luciferase in which the modified luciferase comprises a selectivity for ATP over dATP that is at least three-fold greater than the selectivity for ATP over dATP of an unmodified luciferase from the same organism. In a preferred embodiment, the selectivity for ATP over dATP is at least eight-fold greater than the selectivity for ATP over dATP of an unmodified luciferase from the same organism. In more preferred embodiments, the selectivity for ATP over dATP is at least ten-fold greater then the selectivity for ATP over dATP of an unmodified luciferase from the same organism. In even more preferred embodiments, the selectivity for ATP over dATP is at least fifteen-fold greater then the selectivity for ATP over dATP of an unmodified luciferase from the same organism.

More embodiments of the present invention include cells transformed with a nucleic acid encoding a modified luciferase in which the modified luciferase comprises a selectivity for ATP over dATP that is at least three-fold greater than the selectivity for ATP over dATP of an unmodified luciferase from the same organism. In a preferred embodiment, the selectivity for ATP over dATP is at least eight-fold greater than the selectivity for ATP over dATP of an unmodified luciferase from the same organism. In more preferred embodiments, the selectivity for ATP over dATP is at least ten-fold greater then the selectivity for ATP over dATP of an unmodified luciferase from the same organism. In even more preferred embodiments, the selectivity for ATP over dATP is at least fifteen-fold greater then the selectivity for ATP over dATP of an unmodified luciferase from the same organism.

More embodiments of the present invention include methods for making a modified luciferase comprising a selectivity for ATP over dATP that is at least three-fold greater than the selectivity for ATP over dATP of an unmodified luciferase from the same organism, in which the methods include obtaining a nucleic acid encoding a luciferase; altering the nucleic acid, thereby increasing the selectivity of the luciferase encoded by the nucleic acid and expressing said nucleic acid, thereby making said modified luciferase having a selectivity for ATP over dATP that is at least three-fold greater than the selectivity for ATP over dATP of an unmodified luciferase. In a preferred embodiment, the selectivity for ATP over dATP is at least eight-fold greater than the selectivity for ATP over dATP of an unmodified luciferase from the same organism. In more preferred embodiments, the selectivity for ATP over dATP is at least ten-fold greater then the selectivity for ATP over dATP of an unmodified luciferase from the same organism. In even more preferred embodiments, the selectivity for ATP over dATP is at least fifteen-fold greater then the selectivity for ATP over dATP of an unmodified luciferase from the same organism.

In some embodiments of the above-described methods, altering the nucleic acid comprises introducing an amino acid substitution into the polypeptide encoded by the nucleic acid.

In some embodiments of the above-described methods, the modified luciferase further comprises a light emitting activity that is greater than the light emitting activity of the unmodified luciferase.

In some embodiments of the above-described methods, the modified luciferase is from Photinus pyralis.

In some such embodiments, where an amino acid substitution is introduced, the amino acid substitution is selected from the group consisting of N197, S198, H244, 1423, and any combination thereof. In some such embodiments, the amino acid substitution is selected from the group consisting of T214A, I232A, F295L, E354K, and any combination thereof.

More embodiments of the present invention include methods of obtaining nucleic acid sequence information, the methods comprising providing a nucleotide to a target nucleic acid in the presence of a polymerase; and detecting incorporation of the nucleotide into a polynucleotide complementary to the target nucleic acid by detecting light emitted from a reaction mediated by a modified luciferase, the modified luciferase comprising a selectivity for ATP over dATP that is greater than the selectivity for ATP over dATP of an unmodified luciferase from the same organism. In a preferred embodiment, the selectivity for ATP over dATP is at least three-fold greater than the selectivity for ATP over dATP of an unmodified luciferase from the same organism. In another preferred embodiment, the selectivity for ATP over dATP is at least eight-fold greater than the selectivity for ATP over dATP of an unmodified luciferase from the same organism. In more preferred embodiments, the selectivity for ATP over DATP is at least ten-fold greater then the selectivity for ATP over dATP of an unmodified luciferase from the same organism. In even more preferred embodiments, the selectivity for ATP over dATP is at least fifteen-fold greater then the selectivity for ATP over dATP of an unmodified luciferase from the same organism.

In some embodiments of the above described methods of obtaining nucleic acid sequence information, the nucleotide comprises dATP. In other embodiments of the above described methods of obtaining nucleic acid sequence information, dATPαS is not provided in place of dATP.

In some embodiments of the above described methods of obtaining nucleic acid sequence information, the reaction mediated by modified luciferase comprises the conversion of luciferin and ATP to oxyluciferin, AMP and light, wherein ATP is produced by the reaction of adenylyl sulfate (APS) and pyrophosphate.

In some embodiments of the above described methods of obtaining nucleic acid sequence information, the modified luciferase comprises an altered amino acid sequence as compared to the amino acid sequence of an unmodified luciferase from the same organism.

In some embodiments of the above described methods of obtaining nucleic acid sequence information, the modified luciferase comprises an altered amino acid sequence, the modified luciferase is from Photinus pyralis.

In some embodiments of the above described methods of obtaining nucleic acid sequence information in which the modified luciferase comprises an altered amino acid sequence, the altered amino acid sequence comprises an amino acid substitution. In some such embodiments, the amino acid substitution is selected from the group consisting of N197, S198, H244, 1423 and any combination thereof. In some such embodiments, the amino acid substitution is selected from the group consisting of N197F, S198T, H244F, I423Y and any combination thereof. In some such embodiments, the modified luciferase comprises a greater thermostability than said unmodified luciferase. In some such embodiments, the modified luciferase further comprises an amino acid substitution selected from the group consisting of T214, I232, F295, E354, and any combination thereof. In some such embodiments, the modified luciferase further comprises an amino acid substitution selected from the group consisting of T214A, I232A, F295L, E354K, and any combination thereof.

In some embodiments of the above described methods of obtaining nucleic acid sequence information in which the altered amino acid sequence comprises an amino acid substitution, the modified luciferase further comprises a biotin binding moiety. In some such methods, the amino acid substitution is selected from the group consisting of T214A, I232A, F295L, E354K, N197F, S198T, H244F, I423Y and any combination thereof.

In some embodiments, of the above-described methods of obtaining nucleic acid sequence information, the modified luciferase is associated with a solid support. In some such methods, the solid support comprises a particle. In other embodiments, the solid support comprises a well. In still other embodiments, the solid support comprises a planar surface.

In some embodiments, of the above-described methods of obtaining nucleic acid sequence information, detecting incorporation of the nucleotide into a polynucleotide complementary to the target nucleic acid is carried out at a temperature of 20-40° C.

In some embodiments, of the above-described methods of obtaining nucleic acid sequence information, detecting incorporation of said nucleotide into a polynucleotide complementary to said target nucleic acid is carried out at a pH of 5.5-9.

More embodiments of the present invention include apparatuses for obtaining nucleic acid sequence information. In some such embodiments, an apparatus can include a chamber comprising a target nucleic acid associated with a substrate; a polymerase in fluid communication with said nucleic acid, and a modified luciferase in fluid communication with said target nucleic acid, wherein said modified luciferase comprises a selectivity for ATP over dATP that is greater than the selectivity for ATP over dATP of an unmodified luciferase from the same organism. In a preferred embodiment, the selectivity for ATP over dATP is at least three-fold greater than the selectivity for ATP over dATP of an unmodified luciferase from the same organism. In another preferred embodiment, the selectivity for ATP over dATP is at least eight-fold greater, than the selectivity for ATP over dATP of an unmodified luciferase from the same organism. In more preferred embodiments, the selectivity for ATP over dATP is at least ten-fold greater then the selectivity for ATP over dATP of an unmodified luciferase from the same organism. In even more preferred embodiments, the selectivity for ATP over dATP is at least fifteen-fold greater then the selectivity for ATP over dATP of an unmodified luciferase from the same organism.

In some embodiments of the above-described apparatuses, the modified luciferase comprises an altered amino acid sequence as compared to the amino acid sequence of an unmodified luciferase from the same organism.

In some embodiments of the above-described apparatuses, the modified luciferase is from Photinus pyralis. In some embodiments, the altered amino acid sequence of the modified luciferase comprises an amino acid substitution. In certain embodiments, the amino acid substitution is selected from the group consisting of N197, S198, H244, 1423 and any combination thereof. In some such embodiments, the amino acid substitution is selected from the group consisting of N197F, S198T, H244F, I423Y and any combination thereof.

In some embodiments of the above-described apparatuses, the modified luciferase comprises a greater thermostability than said unmodified luciferase. In some such embodiments, the modified luciferase further comprises an amino acid substitution selected from the group consisting of T214, I232, F295, E354, and any combination thereof. In other such embodiments, the modified luciferase further comprises an amino acid substitution selected from the group consisting of T214A, I232A, F295L, E354K, and any combination thereof.

In some embodiments of the above-described apparatuses comprising a modified luciferase having an amino acid substitution, the modified luciferase further comprises a biotin binding moiety. In some such embodiments, the amino acid substitution is selected from the group consisting of T214A, I232A, F295L, E354K, N197F, S198T, H244F, I423Y and any combination thereof.

In some embodiments of the above-described apparatuses, the modified luciferase is associated with a solid support. In some embodiments, the solid support comprises a particle. In some embodiments, the solid support comprises a well. In some embodiments, the solid support comprises a planar surface.

Some embodiments of the above-described apparatuses further comprise a detector of emitted light coupled to the chamber.

Additional embodiments of the present invention are presented below. These embodiments relate to engineered luciferase enzymes, nucleic acids encoding luciferase functional mutant proteins of luciferase, methods of sequencing, in particular pyrosequencing, using engineered luciferase enzymes and methods for producing engineered luciferases. Accordingly, one aspect of the invention is an engineered luciferase enzyme that is modified such that the selectivity of the engineered luciferase toward ATP over dATP that is higher than the selectivity of a luciferase enzyme without the modifications.

In one embodiment, the modification to the enzyme comprises a chemical modification to the enzyme. In one embodiment, the chemical modification comprises chemical coupling or cross-linking of the target enzyme. In one embodiment, the enzyme is derived from Photinus pyralis. In one embodiment, the modification comprises one or more mutations. In one embodiment, the modification comprises changing one or more of residues corresponding to R437, D422, R537, 1423, D436, and L530 of Photinus pyralis luciferase. In one embodiment, the one or more mutations comprise one or more corresponding to I423L, D436G, and L530R of Photinus pyralis luciferase. In one embodiment, the mutations comprise two or more of corresponding to I423L, D436G, and L530R of Photinus pyralis luciferase. In one embodiment, the mutations comprise at least the mutations corresponding to I423L, D436G, and L530R of Photinus pyralis luciferase. In one embodiment, the luciferase enzyme without the modifications is a wild type luciferase. In one embodiment, the luciferase enzyme without the modifications is a mutated luciferase. In one embodiment, the luciferase enzyme is a thermostable luciferase. In one embodiment, the modification comprises changing one or more of residues corresponding to amino acids selected from the group consisting of T214, I232, F295, E354, N197, S198, H244, I423 and any combination thereof. In some such embodiments, the modification comprises a modification selected from the group consisting of T214A, I232A, F295L, E354K, N197F, S198T, H244F, I423Y and any combination thereof.

In one embodiment, the selectivity of the engineered enzyme is more than 20% higher than the selectivity of the enzyme without the modifications. In one embodiment, the selectivity of the engineered enzyme is more than 50% higher than the selectivity of the enzyme without the modifications. In one embodiment, the selectivity of the engineered enzyme is more than 2 times higher than the selectivity of the enzyme without the modifications. In one embodiment, the selectivity of the engineered enzyme is more than 5 times higher than the selectivity of the enzyme without the modifications. In one embodiment, the selectivity of the engineered enzyme is more than 2 times higher than the selectivity of the enzyme without the modification. In one embodiment, the K_{M} for ATP is lower than for the enzyme without mutations. In one embodiment, the K_{M} for ATP is lowered by more than 20%. In one embodiment, the K_{M} for ATP is lowered by more than 50%. In one embodiment, the K_{M} for ATP is lowered by more than 2 times. In one embodiment, the K_{M} for ATP is lowered by more than 5 times. In one embodiment, the K_{M} for ATP is 45 μM or less. In one embodiment, the K_{M} for ATP is 10 μM or less. In one embodiment, the mutation would change the charge, hydrophobicity, hydrophilicity, or size of the ATP binding pocket on the enzyme.

One aspect of the invention is a functional mutant protein comprising an amino acid sequence that differs from the sequence of a Photinus pyralis luciferase protein sequence by at least an amino acid substitution at residues corresponding to R437, D422, R537, I423, D436, or L530 of Photinus pyralis luciferase, wherein said mutant protein has a higher selectivity for ATP over dATP than the a Photinus pyralis luciferase protein. In one embodiment, the substitution comprises mutations that correspond to I423L, D436G, or L530R of Photinus pyralis luciferase.

One aspect of the invention is a nucleic acid molecule comprising a nucleic acid sequence encoding a functional mutant protein whose amino acid sequence differs from that of a Photinus pyralis luciferase protein sequence by at least an amino acid substitution selected from the group consisting of substitutions corresponding to I423L, D436G, and L530R of Photinus pyralis luciferase, said mutant protein having a higher selectivity for ATP over dATP than a Photinus pyralis luciferase protein.

One aspect of the invention is method of sequencing that uses all natural deoxy nucleoside triphosphates.

One aspect of the invention is a method of pyrosequencing that uses all natural deoxy nucleoside triphosphates.

In one embodiment, the invention is a method wherein dATP, dTTP, dGTP, and dCTP are used. In one embodiment, the invention is a method wherein no dATPαS is used. In one embodiment, the invention is a method wherein dATP, dTTP, dGTP, and dCTP are used and no dATPαS is used.

In one embodiment, the method uses a luciferase enzyme and the luciferase enzyme has a selectivity for ATP over dATP of greater than 100:1. In one embodiment, the method uses a luciferase enzyme and the luciferase enzyme has a selectivity for ATP over dATP of greater than 200:1. In one embodiment, the method uses a luciferase enzyme and the luciferase enzyme has a selectivity for ATP over dATP of greater than 500:1.

In one embodiment, the sequencing reaction is carried out at a temperature of 20-37° C. In one embodiment, the sequencing reaction is carried out at a pH of 6-9. In one embodiment, an engineered luciferase enzyme is used. In one embodiment, the engineered luciferase comprises mutations at one or more of residues corresponding to R437, D422, R537, I423, D436, and L530 of Photinus pyralis luciferase. In one embodiment, the engineered luciferase comprises one or more of the mutations corresponding to I423L, D436G, and L530R of Photinuspyralis luciferase.

One aspect of the invention is a method of pyrosequencing wherein the error rate when sequencing a homopolymeric region having multiple, for example 4, ‘A’ nucleotides is less than when dATPαS is used.

One aspect of the invention is a method for producing an engineered luciferase enzyme comprising performing site directed mutagenesis to produce an enzyme wherein the selectivity of the engineered luciferase toward ATP over dATP that is higher than the selectivity of a luciferase enzyme without the modifications having mutations such that the engineered luciferase. In one embodiment, the modification comprises changing one or more of residues corresponding to R437, D422, R537, I423, D436, and L530 of Photinus pyralis luciferase. In one embodiment, the one or more mutations comprise one or more of mutations corresponding to I423L, D436G, and L530R of Photinuspyralis luciferase. In one embodiment, the mutations comprise two or more of residues corresponding to I423L, D436G, and L530R of Photinus pyralis luciferase. In one embodiment, the mutations comprise at least the mutations corresponding to I423L, D436G, and Leu530Arg of Photinus pyralis luciferase. In other embodiments, the modification comprises changing one or more of residues corresponding to amino acids selected from the group consisting of T214, I232, F295, E354, N197, S198, H244, I423 and any combination thereof. In some such embodiments, the modification comprises a modification selected from the group consisting of T214A, I232A, F295L, E354K, N197F, S198T, H244F, I423Y and any combination thereof.

### BRIEF DESCRIPTION OF THE DRAWINGS

FIG. 1 shows an alignment of the amino acid sequence (SEQ ID NO: 5-15) for luciferases from various organisms (Lcr, Lla, Lmi, Pmi, Ppy, Lno, Ppel, Phg, GR, YG, Ppe2, respectively). The sequences are aligned, spaces where sequences cannot be aligned are shown by dots ( . . . ), ‘Cons’ shows conserved amino acids and indicates non-conserved amino acids by “−”.

FIG. 2 is a schematic of a model of the Photinus pyralis luciferase active site.

FIG. 3 is a diagram illustrating the effect of dATP and dATPαS on luciferase reaction.

FIG. 4 is bar chart showing a comparison of the enzymatic activity of wild-type and mutated Photinuspyralis luciferase using dATP or ATP as substrates.

FIG. 5 is a diagram illustrating solid-phase pyrosequencing with SEQ ID NO:16.

FIG. 6 is a diagram illustrating liquid-phase pyrosequencing with SEQ ID NO:16.

FIG. 7A shows a bar chart of integrated relative light units (RLU) in light emission assays for various luciferases in the presence of 0.5 mM ATP. FIG. 7B shows a bar chart of peak RLU in light emission assays for various luciferases in the presence of 0.5 mM ATP.

FIG. 8A shows a bar chart of integrated relative light units (RLU) in light emission assays for various luciferases in the presence of 0.05 μM ATP. FIG. 8B shows a bar chart of peak RLU in light emission assays for various luciferases in the presence of 0.05 μM ATP.

FIG. 9 shows a bar chart of ratios of ATP activity over dATP activity for various luciferases. Ratios were determined from measuring activities using 0.05 μM ATP/0.5 mM dATP, or using several concentrations of ATP/dATP and averaging the results.

FIG. 10 shows a plot of RLU over time for a modified luciferase associated with a bead.

### DETAILED DESCRIPTION

Some embodiments of the present invention relate to modified luciferases having a selectivity for ATP over dATP that is greater than the selectivity for ATP over dATP of an unmodified luciferase. Other embodiments of the present invention relate to nucleic acids encoding modified luciferases, methods for making modified luciferases, methods for obtaining sequence information, such as pyrosequencing, and apparatuses for obtaining sequence information comprising modified luciferases.

One aspect of the present invention is a luciferase enzyme that has improved selectivity for ATP over dATP. This improved selectivity can be important for carrying out pyrosequencing. Current methods of pyrosequencing generally do not allow the use of dATP as one of the nucleotides in the sequencing reaction because it acts as a substrate for luciferase, resulting in unacceptable background light levels of 1% to 2%. Some aspects of the present invention allow for pyrosequencing using dATP, the natural substrate for the polymerase enzyme. The improved selectivity brings the level of background light generated by dATP as a substrate down to acceptable levels. The use of dATP as a nucleotide for pyrosequencing rather than dATPαS provides for more accurate sequencing because dATP is a better substrate for the polymerase enzyme than is dATPαS, resulting in faster, more accurate incorporation. Certain embodiments of the invention provide in an improvement to current pyrosequencing methods incorporating dATPαS especially in sequencing homopolymeric regions in which multiple ‘A’s are incorporated sequentially.

In one embodiment, an engineered luciferase enzyme is modified such that the selectivity of the engineered luciferase toward ATP over dATP is higher than the selectivity of a luciferase enzyme without the modifications.

Engineered luciferase enzymes described herein can be derived from any luciferase enzyme. Examples of luciferase enzymes from which the engineered enzymes described herein can be generated are: Japanese GENJI and HEIKE fireflies Luciola cruciata and Luciola lateralis, the East European Firefly Luciola mingrelica, the North American firefly (Photinus pyralis), Photuris pennsylvanica (Genbank: D25416.1; GI:1669525), the glow-worm and the European glow-worm Lampyris noctiluca, the Iranian firefly Lampyris turkestanicus, and species of Brazilian fireflies in the genera: Phorinus, Photinoides, Macrolampis, Aspisoma, Cratomorphus, Amydetes, Photuris, Bicellonychia, Pyrogaster. Genebank accession numbers for some of these luciferases include: Luciola cruciata luciferase (P13129, protein; M26194, DNA); Luciola lateralis luciferase (Q01158, protein; X66919, DNA); Luciola mingrelica luciferase, (AAB26932, protein; S61961, DNA); Photinus pyralis luciferase (AAA29795, protein; M15077, DNA); Lampris noctiluca luciferase (AAW72003, protein; AY748894, DNA).

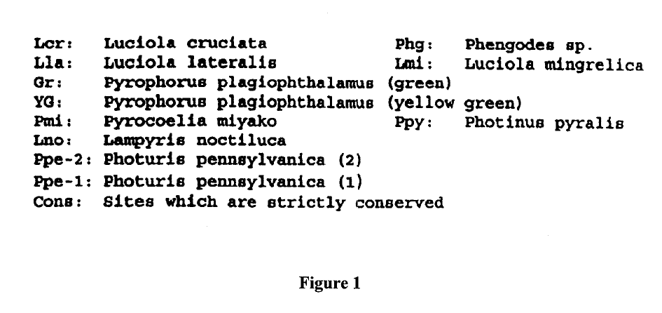

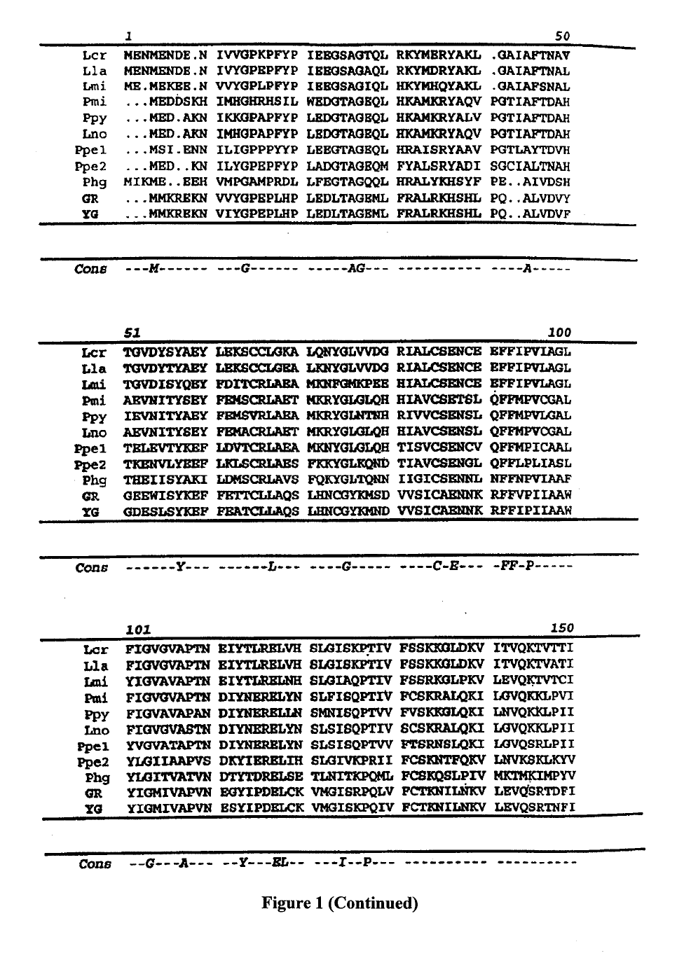

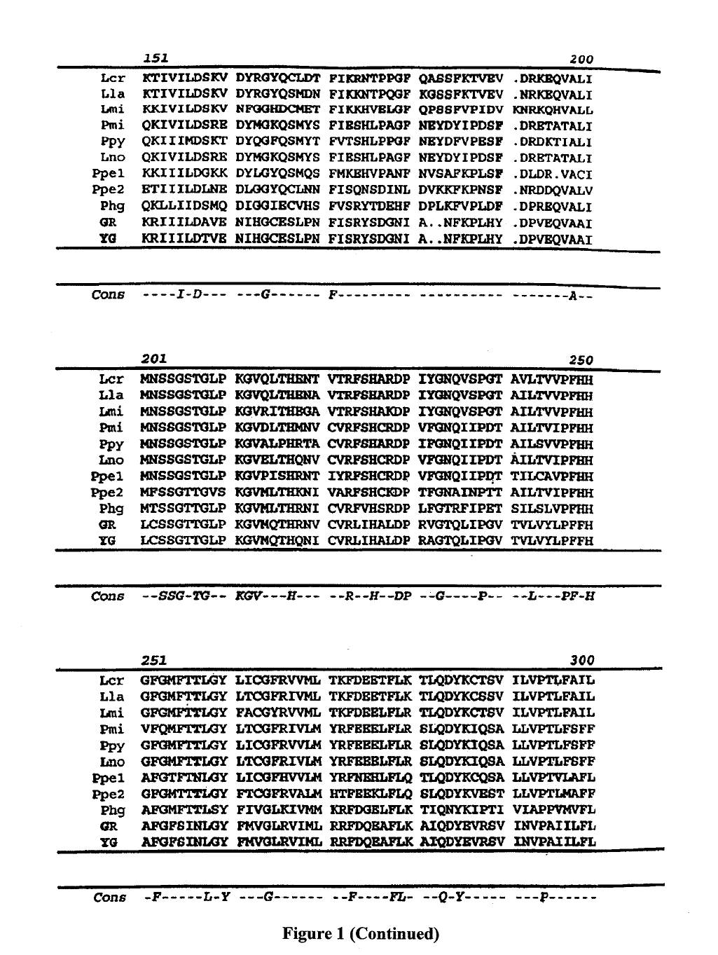

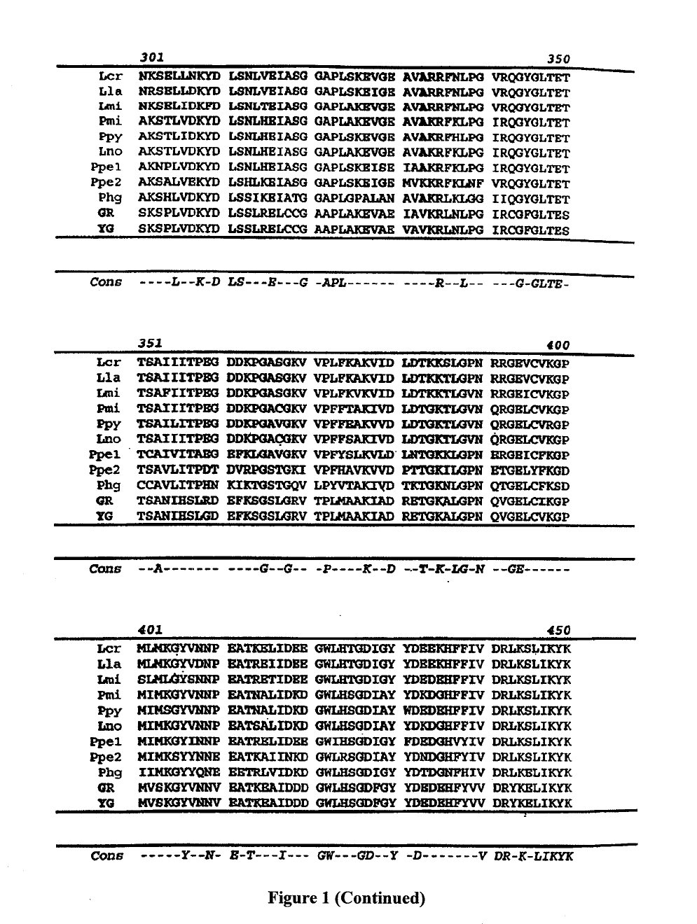

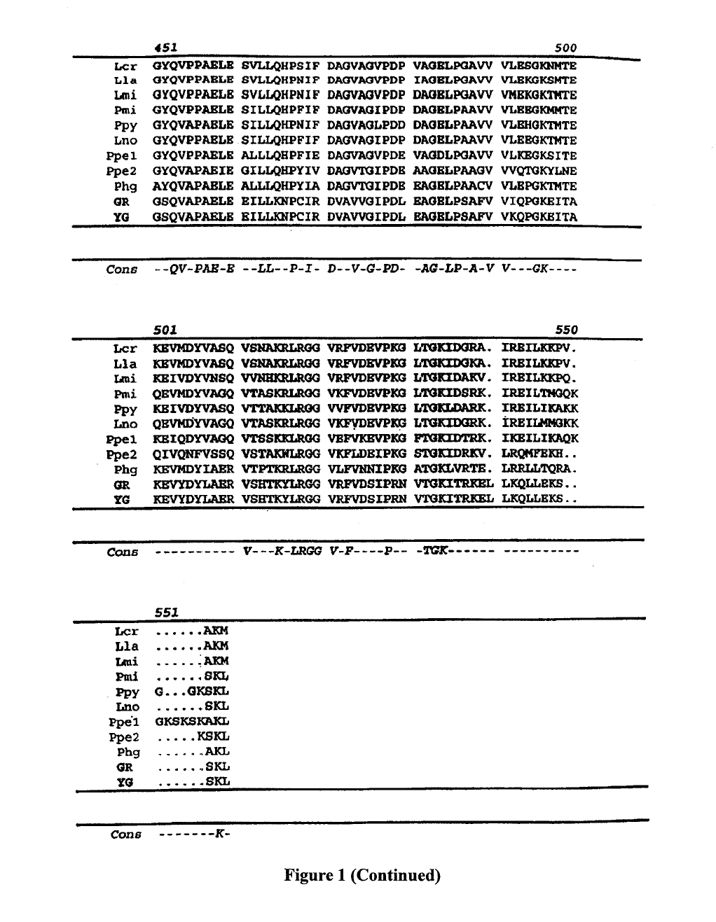

Luciferases are well conserved across species. FIG. 1 illustrates examples of selected regions of various luciferases where primary sequences align (see, for example, Wood, K. V., et al. (1989) Science 244, 700-702; Ye, L., et al. (1997) Biochim. Biophys. Acta 1339, 39-52; Viviani, V. R., et al. (1999) Biochemistry 38, 8271-8279; Viviani, V. R., et al. (1999) Photochem. Photobiol. 70, 254-260; DeWet, J. R., et al. (1987) Mol. Cell. Biol. 7, 725-737; Sala-Newby, G. B., et al. (1996) Biochem. J. 313, 761-767; Ohmiya, Y., et al. (1995) Photochem. Photobiol. 62, 309-313; Tatsumi, H., et al. (1992) Biochim. Biophys. Acta 1131, 161-165; Cho, K. H. (1995) GenBank Z49891, direct submission; Masuda, T., et al. (1989) Gene 77, 265-270; Devine, J. H., et al. (1993) Biochim. Biophys. Acta 1173, 121-132). Accordingly, some of the modified luciferases described herein include luciferases from various organisms. In particular embodiments, modifications exemplified herein for a luciferase from a particular organism can be made at equivalent positions in a luciferase from one or more other organisms. It will be understood that equivalent positions are those that are aligned to each other in a primary sequence alignment for the luciferases from two or more organisms, or those positions that are identified as equivalent via protein modelling methods based on tertiary structure predictions, threading, and/or energy minimization analyzes.

Methods to compare, align, and identify primary, secondary, and tertiary sequences of proteins are well known in the art. In more embodiments, methods can be used to identify consensus sequences between different proteins, such as between different types of proteins, proteins from different organisms. In more embodiments, methods can be used to identify common structure and sites between different proteins. Examples of sequence analysis software includes the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Additionally, Secondary and tertiary structures can be compared by methods such as aligning the predicted structures, for example, by superimposing predicted structures on one another. Examples of software that may be used include Swizz-PdbViewer (Swizz Institute for Bioinformatics), TOPOFIT (Valentin A. et al, Protein Science (2004), 13:1865-1874), SOLVX (Holm L, Sander C (1992) J Mol Biol 225(1):93-105), DALI (L Holm, C Sander (1993) J Mol Biol 233(1): 123-38. S Dietmann, et al (2001). Nucleic Acids Res 29(1): 55-7), and MaxSprout (L Holm, C Sander (1991) J Mol Biol 218(1): 183-194.), and Vector Alignment Search Tool (VAST) (National Center for Biotechnology Information).

### Selectivity

Selectivity of a luciferase toward ATP or dATP can be assessed by measuring the light emitting activity of the luciferase. The detection of light using luciferin as a substrate can be used. The activity of luciferase can be described in light units, for example, Units/mg solid, or Units/mg protein. For example, light units are measured in 50 μl of assay mixture containing 5 μmol of ATP and 7.5 nmol luciferin in glycine Tris buffer, pH 7.6 at 25° C. Under these conditions, one light unit produces a biometer peak height equivalent to 0.02 μCi of ¹⁴C in 2,5-Diphenyloxazole (PPO)/1,4-bis[5-Phenyl-2-oxazolyl]-benzene (POPOP).

PPO/POPOP cocktail (Leach, F. R. and Webster, J. J. (1986) Methods in Enzymology, 133, Part B, 51-70; Lin, S, and Cohen, H. P. (1968) Analytical Biochemistry 24, 531-540 and Strehler, B. L. (1974) in Methods of Enzymatic Analysis (Bergmeyer, H. U. ed.) 2nd ed., Vol. 4, 2112-2121, the disclosures of which are incorporated herein by reference in their entireties).

Generally, the selectivity is measured by running a pair of reactions, wherein in one reaction, ATP is the substrate, and in the other, dATP is the substrate. The pair of reactions is generally run such that all conditions are the same. In some cases the concentration of the dATP and the ATP are not the same in the paired reactions, but the concentration levels are adjusted in order to measure adequate light output. In these cases, the results are adjusted for concentration in order to obtain the selectivity. Activity assays can be performed under a variety of conditions. Variables that can be altered in an activity assay include time, temperature, pH, salt concentrations, detergent concentrations, buffers, and buffer concentrations. Buffers can include Tris, tricine, HEPES, TES, MOPS, PIPES, cacoylate, MES, acetate, phosphate, and citrate. Buffers are well known by those skilled in the art and are available from, for instance, Sigma. Reactions can be performed at a range of temperatures. Reactions to measure selectivity can be performed at 20° C.-40° C. Reactions can be performed at about 25° C., 30° C., and 37° C.

Some modified luciferases described herein can have a selectivity for ATP over dATP compared to a selectivity for ATP over dATP of an unmodified luciferase that is greater by a factor of at least about 2, 3, 4, 5, 6, 7, 8, 9, 10, 11, 12, 13, 14 or 15.

Some modified luciferases described herein can have a light emitting activity that is greater than the light emitting activity of an unmodified luciferase. In some embodiments, the light emitting activity of a modified luciferase can be greater than the light emitting activity of an unmodified luciferase by a factor of at least about 2, 3, 4, 5, 6, 7, 8, 9, 10, 11, 12, 13, 14, 15, 16, 17, 18, 19, and 20. In some embodiments, modified luciferases described herein can have both an increased selectivity for ATP over dATP compared to a selectivity for ATP over dATP of an unmodified luciferase and a light emitting activity that is greater than the light emitting activity of an unmodified luciferase.

### Chemical Modifications

A modification can be a chemical modification. A chemical modification can be chemical coupling. A chemical coupling generally involves reacting the enzyme with a chemical reactant that adds to the enzyme, adding a moiety to the enzyme. For example, the chemical coupling can comprise the addition of an acetyl group. The reaction of proteins with groups that modify the properties of the protein are well known in the art. In some cases, the modification involves adding substituents to the amino groups of the lysine residues of the luciferase. In some cases, these modifications can result in the size of the pocket of the active site of the enzyme, thus improving specificity. It will be appreciated that chemical modification can be covalent or non-concovalent. In preferred embodiments, the modification is a covalent modification.

A chemical modification can involve cross-linking. Cross-linking can be within one luciferase molecule (intramolecular cross-linking). Cross-linking can be between luciferase and another entity or with another luciferase molecule. Cross-linking can occur in vitro or in vivo. Cross-linking can be by homobifunctional or heterobifunctional crosslinkers. Cross-linking can be by photoreactive crosslinkers. Cross linking can be performed with any chemical molecule carrying two active groups. Chemical molecules can be of different length resulting in different cross-linked protein. Cross-linking can be by one or more disulfide bonds. Cross-linking can be achieved by using imidoester crosslinker dimethyl suberimidate, the NHS-ester crosslinker BS3 and formaldehyde, AEDP, ASBA (4-[p-Azidosalicylamido]butyamine), DCC, EDC (1-Ethyl-3[3-dimethylaminopropyl]carboiimide hydrochloride), ANB-NOS (N-5-Azido-2-nitrobenzoyloxysuccinimide), NHS-ASA (N-Hydroxysuccinimidyl-4-azidosalicylic acid), SADP (N-Succinimidy1(4-azidopheny1)-1,3′-dithiopropionate, SAND (Sulfosuccinimidyl 2 μm-azido-o-nitrobenzamido]-ethyl-1,3′-dithiopropionate), SANPAH (N-Succinimidyl-6-[4′-azido-2′-nitrophenylamino]hexanoate), sulfo-HSAB (N-Hydroxysulfosuccinimidyl-4-azidobenzoate), Sulfo-NHS-LC-ASA (Sulfosuccinimidy1 [4-azidosalicylamido]-hexanoate), Sulfo-SADP (N-Sulfosuccinimidy1(4-azidophenyl)-1,3′-dithiopropionate), Sulfo-SAED (Sulfosuccinimidyl 247-amino-4-methylcoumarin-3-acetamidoiethyl-1,3′dithiopropionate), Sulfo-SANPAH (N-Sulfosuccinimidyl-6[4′-azido-2′-nitrophenylamino]hexanoate), Sulfo-SBED (Sulfo-N-hydroxysuccinimidyl-2-(6-[biotinamido]-2-(p-azido benzamido)-hexanoamido) ethyl-1,3′-dithioproprionate), Sulfo-SFAD (Sulfosuccinimidyl-[perfluoroazidobenzamido]ethyl-1,3′-dithiopropionate), Sulfo-SMCC, and gluteraldehyde. Other cross-linkers are well known by those skilled in the art and are available, for instance, from Thermo Scientific.

Another chemical modification useful for the modification of luciferase can be a posttranslational modification. The chemical modification can be carried out by an enzyme or chemically. Examples of post-translational modifications include acetylation, methylation, amidation, biotinylation, formylation, gamma-carboxylation, glutamylation, glycosylation, glycylation, hydroxylation, iodination, isoprenylation, prenylation, myristoylation, farnesylation, geranylgeranylation, ADP-ribosylation, flavin attachment, oxidation, palmitoylation, pegylation, phosphatidylinositol attachment, phosphopantetheinylation, phosphorylation, polysialyation, pyroglutamate, racemization of proline, arginylation, sulfation, selenoylation, sulfation, ISGylation, ubiquitination, sumolyation, citrullination (deimination), and deamidation. Posttranslational modifications are well known by those skilled in the art.

### Mutations

In some embodiments of the present invention, modified luciferases described herein can comprise one or more mutations. Mutations can include additions, insertions, deletions, and substitutions of at least one amino acid in a luciferase. Furthermore, the mutations can be in the amino acids. The amino acid can be in the L-isomeric form. When an amino acid residue is part of a polypeptide chain, the D-isomeric form of the amino acid can be substituted for the L-amino acid residue, as long as the desired functional property is retained.

Amino acids can be represented by their standard 1-letter code or 3-letter code. An amino acid residue represented by “X” or “Xxx” refers to any one of the naturally occurring or non-naturally occurring amino acid residues known in the art or to a modification of a nearby residue. In keeping with standard protein nomenclature, all amino acid residue sequences represented herein by formulae have a left to right orientation in the conventional direction of amino-terminus to carboxyl-terminus. Watson et al., book (1987, Molecular Biology of the Gene, 4th Edition, The Benjamin Cummings Pub. Co., p. 224), incorporated herein by reference. Amino acid substitutions are typically of single residues, but may be of multiple residues, either clustered or dispersed. An amino acid can be replaced with a different naturally occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Additions encompass the addition of one or more naturally occurring or non-conventional amino acid residues. Deletion encompasses the deletion of one or more amino acid residues. Variant polypeptides may contain conservative amino acid substitutions at various locations along their sequence, as compared to the R polypeptide amino acid sequences of the invention. A “conservative amino acid substitution” is one in which the amino acid residue is replaced with an amino acid residue having a similar side chain.

As is known in the art, families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

In some embodiments, substitutions may be “non-conservative”, in which an amino acid residue which is present in a peptide is substituted with an amino acid having different properties, such as naturally-occurring amino acid from a different group (e.g., substituting a charged or hydrophobic amino acid with alanine), or alternatively, in which a naturally-occurring amino acid is substituted with a non-conventional amino acid.

Mutations can be made using chemicals or radiation. Chemicals used for mutagenesis can include ethyl methane sulfonate (EMS), methyl methane sulfonate (MMS), diethylsulfate (DES), and nitrosoguanidine (NTG, NG, MNNG), nitrous acid, 5-bromo-deoxyuridine (5BU), ethidium bromide. Radiation for mutagenesis can include ultraviolet, ionizing, or gamma. Mutations can be made using polymerase chain reaction (PCR). Mutations can be generated randomly. Methods of mutagenesis are well known by those of skill in the art.

Modifications can be introduced by cloning methods using cloning vectors known in the art. Modifications can include mutations that change, for example one or more of Arg437, Asp422, Arg537, Ile423, Asp436, and Leu530 in Photinus pyralis luciferase. In some embodiments, the substitutions can include one or more substitutions of T214, I232, F295, E354, N197, S198, H244, I423, or any combination thereof. The mutations can be made in the corresponding residues in luciferases from other organisms.

The mutations can be one or more, two or more, or at least the three mutations Ile423Leu, Asp436Gly, and Leu530Arg in a Photinus pyralis luciferase. In more embodiments, the substitutions can be at least one selected from T214A, I232A, F295L, E354K, N197F, S198T, H244F, I423Y, or any combination thereof. The corresponding mutation in luciferases from other organisms can be mutated. Identifying the site of a corresponding mutation can be carried out by mapping the sequences of the enzymes to identify the corresponding amino acid sites on the proteins. Such mapping is known in the art, such as through sequence alignments.

In some embodiments, a luciferase enzyme can be a wild-type or mutated luciferase enzyme. Mutated luciferase enzymes with, for example, higher thermal stability and with modified light color have been created. In some embodiments, these enzymes are engineered in order to produce an enzyme with both the improved property, such as thermal stability or light color, and improved ATP/dATP selectivity over the non-engineered enzyme. A thermostable luciferase can be a wild-type or mutated luciferase. Thermostable luciferases are known in the art (see, for example, Tisi et al (2002) “Development of thermostable firefly luciferase.” Analytica Chim Acta. 457:115-123, Kajiyama et al. U.S. Pat. No. 5,229,285; U.S. Pat. No. 6,602,677, and U.S. Pat. No. 7,241,584, incorporated by reference in their entireties. Examples of thermostable mutations include at the following positions in Photinus pyralis luciferase, and the equivalent positions of luciferases derived from other organisms, L214, I232, F295, and E354. In some embodiments, thermostable mutations can include substitutions can include at least one substitution such as T214A, I232A, F295L, or E354K, and any combination thereof.

A luciferase enzyme can be modified by changing one or more of residues corresponding to Arg437, Asp422, Arg537, Ile423, Asp436, and Leu530 in Photinus pyralis luciferase and have a selectivity toward ATP over dATP that is at least 2 times higher than the selectivity toward ATP over dATP of an enzyme without the modification(s).

In some embodiments, an engineered enzyme can have a K_{M} for ATP that is lower than for an enzyme without modifications. The K_{M} for ATP can be lowered, for example by at least 10%, 20%, 30%, 40%, or 50%. A modified luciferase enzyme can have a K_{M} for ATP that is lowered by more than at least 2, 3, 4, or 5 times. A modified luciferase enzyme can have a K_{M} for ATP that is 45 μM or less, or 10 μM or less. The K_{M} can be less than 160 μM or less than 2 μM. For example, high light emitting modified luciferases, such as I423L, Y340D, L530R, L438Y and T345S may possess a lower K_{M} for ATP as compared to an unmodified luciferase from the same organism.

### Luciferase Active Site

A mutated luciferase enzyme can have at least one or more mutations that change the charge, hydrophobicity, hydrophilicity, or size of the ATP binding pocket on the enzyme. Luciferase (Photinus pyralis) is believed to be structurally composed of a large N-terminal active site domain (residues 1-436), a flexible linker (residues 436-440) peptide, and a small C-terminal domain (residues 440-550) facing the N domain. The N- and C-terminal domains face each other and come close enough to sandwich the substrates during the reaction. Several amino acids in luciferase are involved in the pocket for substrate specificity, and these residues include the amino acids residing in ATP binding site, and other domain determining the charge and size of the pocket, such as amino acids in C-terminal domain (around residue 530) and the other domain around residue 435. The concepts of hydrophobicity and hydrophilicity are well known by those skilled in the art.

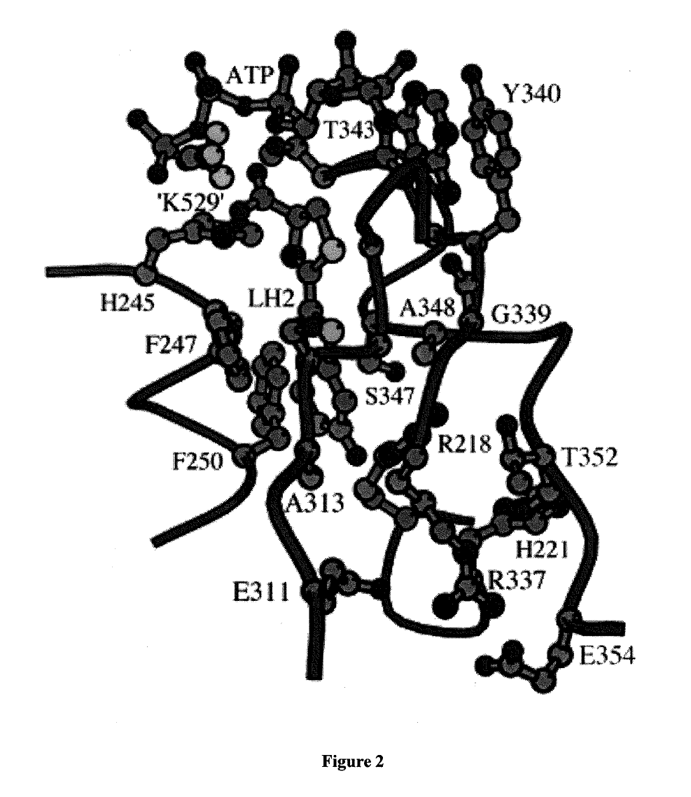

The crystal structure of luciferase is known (see, Conti et al (1996) Structure 4:287-298). FIG. 2 shows an example model of the Luciferase active site containing D-luciferin (LH2) and Mg-ATP, where traces through the α-carbons of regions V217-H221, H244-T252, H310-L319, and R337-G355 are shown. The α-carbons of G246, S314 (and side chain group), G315, G316, and G341 are shown but not labeled. The main chain carbonyl groups of G339 and T352 are also shown. Thr343 is likely part of the adenylate-forming family motif II (³⁴⁰YGTE³⁴⁴) and binds substrates (see, Branchini et al. Biochemistry (2001) 40:2410-2418; and Branchini et al. Biochemistry (1998) 37:15311-15319). Thr527 and Lys529 may be important for effective substrate orientation, forming hydrogen bonds with phosphate groups of ATP, and Lys443 may have a role in AMP release. (Branchini et al Biochemistry (2005) 44:1385-1393). Gly421 and Asp422 form part of the active site and play a role in properly positioning the AMP of luciferyl adenylate. The ribose hydroxyls of ATP form hydrogen bonds to Asp422 and Tyr340, and the adenine moiety of ATP binds to the luciferase cavity formed between residues 315-318 and Ile434 that is located close to Asp436 (Conti et al. (1996) Structure 4:287-298; and Sandlova et al (1999) Biochemistry (Moscow) 64:1143-1150)

More models for the active site of luciferase have been described produced by molecular modeling and energy minimization. (Sandlova et al. (1999) Biochemistry (Moscow) 64:1143-1150). Luciferase may undergo conformational changes during catalysis, where mutations at residues in the distant A8 and A10 motifs both affect the binding of Luciferase substrates D-luciferin, Mg-ATP, and D-luciferyl-AMP (Branchini et al (2005) 44:1385-1393).

Three dimensional structures of luciferases including the active site can readily be determined using software such as BioPackage structural analysis software (MolSoft), BobScript, POVScript+, and Raster 3D (Kraulis P. J. (1991) J. Appl. Crystallogr. 24:946-950; Esnouf R. M. (1999) Acta. Crystallogr. Sect D 55:938-940; Fenn et al. (2003) J. Appl. Crystallogr. 36:944-947; and Merrit et al (1997) Methods Enzymol 277: 505-524).

Models of the active site can include the substrates of luciferase. From such models the distances between particular atoms of residues to substrates of luciferase, such as, dATP, ATP, D-luciferin, and/or D-luciferyl-AMP can readily be determined.

Some modified luciferases described herein can include a substitution at a particular residue where at least one atom of the particular residue is within about 1, 2, 3, 4, 5, 6, 7, 8, 9, 10, 11, 12, 13, 14, 15, 16, 17, 18, 19, and 20 Angstroms of at least one atom of the ATP, D-luciferin, and/or D-luciferyl-AMP bound to the active site of the luciferase. In more embodiments, a substitution can be at a particular residue where at least one atom of the particular residue is within about 1, 2, 3, 4, 5, 6, 7, 8, 9, 10, 11, 12, 13, 14, 15, 16, 17, 18, 19, and 20 Angstroms of the active site of the luciferase. In more embodiments, a substitution can be at a particular residue where at least one atom of the particular residue is within about 1, 2, 3, 4, 5, 6, 7, 8, 9, 10, 11, 12, 13, 14, 15, 16, 17, 18, 19, and 20 Angstroms of at least one atom of a particular residue at the active site of a luciferase. In more embodiments, a substitution can be at a particular residue at the active site of luciferase.

As will be appreciated, because the tertiary structure of the active site of luciferase is conserved across organisms, modified luciferases include those modified at equivalent residues in the active site of other organisms.

Mutations can be in at least 1, 2, 3, 4, 5, 6, 7, 8, 9, 10 or more amino acids. A mutation can be in at least one or more of residues corresponding to Arg437, Asp422, Arg537, Ile423, Asp436, or Leu530 of Photinus pyralis luciferase. In more embodiments, a modified luciferase can include at least one substitution of T214, I232, F295, E354, N197, S198, H244, I423, and any combination thereof. A mutation can be made in luciferase enzymes derived from other species.

In more embodiments, a functional mutant luciferase protein can include an amino acid sequence that differs from the sequence of Photinus pyralis protein sequence by at least an amino acid substitution at residues corresponding to Arg437, Asp422, Arg537, Ile423, Asp436, or Leu530 of Photinus pyralis luciferase, wherein said mutant protein has a higher selectivity for ATP over dATP than the Photinus pyralis enzyme. The functional mutant protein can have at least one or more of the substitutions including substitutions corresponding to Ile423Leu, Asp436Gly, or Leu530Arg of Photinus pyralis luciferase. In preferred embodiments, a modified luciferase having even higher selectivity for ATP over dATP compared to the selectivity for ATP over dATP of an unmodified luciferase can include at least one substitution of T214A, I232A, F295L, E354K, N197F, S198T, H244F, I423Y, and any combination thereof.

In yet another embodiment, a nucleic acid molecule can have a nucleic acid sequence encoding a functional mutant protein whose amino acid sequence differs from that of a Photinus pyralis protein sequence by at least an amino acid substitution selected from the group consisting of mutations corresponding to Ile423Leu, Asp436Gly, and Leu530Arg of Photinus pyralis luciferase, said mutant protein having a higher selectivity for ATP over dATP than an unmodified Photinus pyralis enzyme.

Some embodiments of the present invention include nucleic acids encoding the modified luciferases described herein. Such nucleic acids can be isolated nucleic acids. More aspects of the present invention can include vectors comprising a nucleic acid encoding the modified luciferases described herein. More aspects of the present invention can include a cell comprising the nucleic acids described herein.

Some modified luciferases can include binding moieties. Examples of binding moieties include glutathione S-transferase (GST) sequences, and biotin binding moieties. Such binding moieties can be useful, for example, to associate a modified luciferase with a substrate, or to purify a modified luciferase. GST sequences are well known. In some embodiments, a biotin binding moiety can include a polypeptide. One example of a polypeptide that can confer biotin binding is the Biotin carboxyl carrier protein (BCCP). In some embodiments, a biotin binding moiety comprising a polypeptide can have at least about 50%, 60%, 70%, 80%, 90%, 95%, 99% or greater than 99% amino acid identity or amino acid similarity to SEQ ID NO: 4.

Some modified luciferases described herein can be associated with a substrate. Examples of substrates include, but are not limited to, particles (e.g. beads, microspheres), planar surfaces, and wells. The modified luciferase may be associated with a substrate through a variety of methods, for example, by chemical modification, entrapment, or association through a binding moiety, such as, biotin binding moiety, or a GST sequence.

### Methods for Sequencing

Some embodiments include methods of sequencing that use all natural deoxy nucleoside triphosphates. DNA sequencing generally are carried out with at least one non-natural deoxy nucleotide triphosphate. Sequencing methods include, for example, Maxam-Gilbert sequencing (DNA sequencing by chemical degradation), “sequencing by synthesis”, (e.g. Sanger sequencing, dye-termination electrophoretic sequencing, and pyrosequencing) “sequencing by ligation” (e.g. polony sequencing, SOLiD sequencing), “sequencing by hybridization”, closed complex single molecule sequencing, nanoscale fluidic technologies, sequencing by ligation using nano-arrays of single DNA molecules, sequencing using nanopore arrays, and force spectroscopy. More methods of sequencing are described in U.S. Patent Application No. 61/174,968 “Sequencing Methods” filed May 1, 2009; and U.S. Provisional Application No. 61/140,566 entitled “MULTIBASE DELIVERY FOR LONG READS IN SEQUENCING BY SYNTHESIS PROTOCOLS” filed on Dec. 23, 2008., incorporated by reference in their entireties.

In yet more embodiments, methods of pyrosequencing that use all natural deoxy nucleoside triphosphates are included.

Various pyrosequencing methods, including PPi sequencing methods, are described in, for example, WO9813523A1, Ronaghi, et al., 1996. Anal. Biochem. 242: 84-89, and Ronaghi, et al., 1998. Science 281: 363-365 (1998), and U.S. Pat. No. 6,274,320, each incorporated by reference in their entireties.

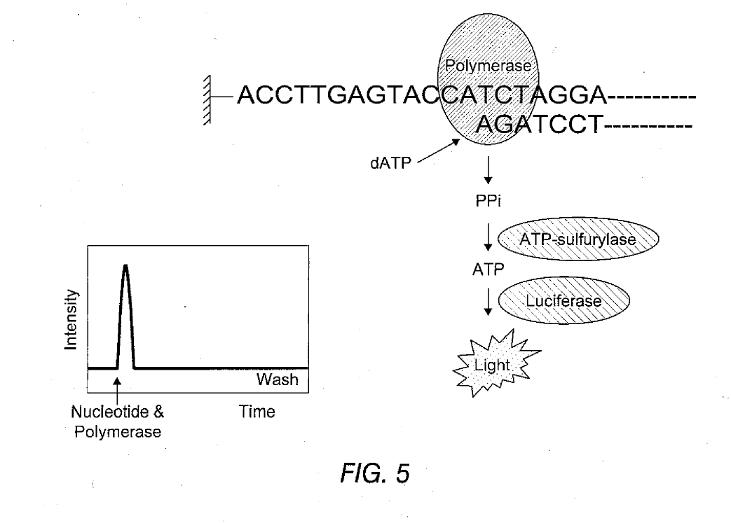

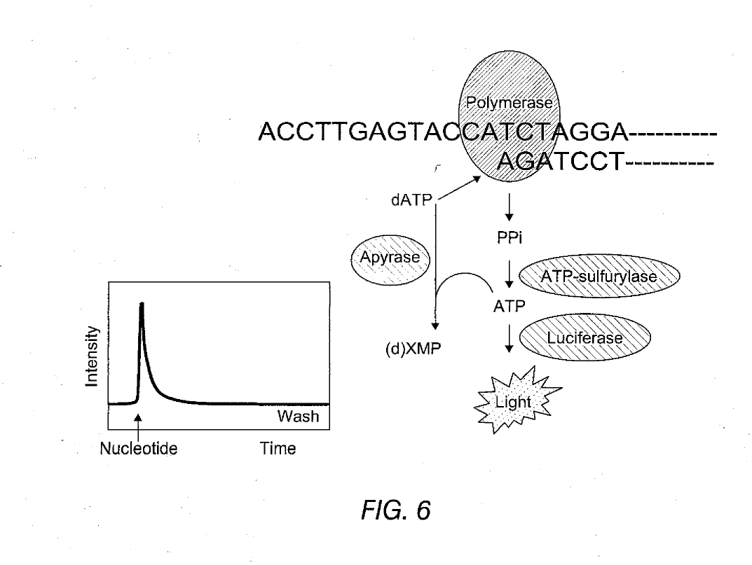

A useful pyrosequencing method is a DNA sequencing technique that is based on the detection of released pyrophosphate (PPi) during DNA synthesis, shown for example in FIG. 5 and FIG. 6. A polymerase catalyzes incorporation of nucleotide(s) into a nucleic acid chain. As a result of incorporation, a pyrophosphate (PPi) molecule(s) is released and subsequently converted to ATP, by ATP sulfurylase. Light is produced in the luciferase reaction during which a luciferin molecule is oxidized.

The natural deoxy nucleoside triphosphates can include dATP, dTTP, dGTP, and dCTP. Because of the increased selectivity for ATP over dATP for certain modified luciferases described herein, in some embodiments of the present invention, pyrosequencing can use dATP without using dATPαS. In some embodiments, the method of pyrosequencing can use dATP, dTTP, dGTP, and dCTP and no dATPαS.

The method of pyrosequencing can use a luciferase enzyme that has a selectivity for ATP over dATP as described highly selective modified luciferases disclosed herein.

In some embodiments, a method of pyrosequencing can be carried out at a temperature at which the selectivity for ATP/dATP is improved. The method of pyrosequencing can be carried out at a temperature of 20-60° C. The temperature can be at about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 53, 54, 55, 56, 57, 58, 59, or 60° C.

In some embodiments, a method of pyrosequencing can be carried out at a pH at which the selectivity for ATP/dATP is improved. The method of pyrosequencing, can be carried out at a pH of 5.5-9. The pH can be at about 5.5, 6.0, 6.5, 7.0, 7.5, 8.0, 8.5, or 9.0.

In some embodiments, a method of pyrosequencing can be carried out at a pH at which the selectivity for ATP/dATP is improved and at a temperature at which the selectivity for ATP/dATP is improved.

In some embodiments, a methods of pyrosequencing can use an engineered luciferase enzyme. For example, in some embodiments, the method of pyrosequencing can use an engineered luciferase that includes mutations at one or more of residues corresponding to Arg437, Asp422, Arg537, Ile423, Asp436, and Leu530 in Photinus pyralis luciferase. In preferred embodiments, a modified luciferase can include at least one substitution selected from T214, I232, F295, E354, N197, S198, H244, I423, or any combination thereof. In some embodiments, methods of pyrosequencing can utilize a modified luciferase described herein. The modifications can be made in corresponding residues in luciferases from other organisms. These residues can be changed in any of the ways described above.

In some embodiments, methods of pyrosequencing can include using an engineered luciferase that includes one or more of the mutations corresponding to Ile423Leu, Asp436Gly, and Leu530Arg in Photinus pyralis. In preferred embodiments, a modified luciferase can include at least one substitution selected from T214A, I232A, F295L, E354K, N197F, S198T, H244F, I423Y, or any combination thereof.

Some methods can be used to detect the sequence of a single molecule, or a homogeneous population of molecules. While the methods can be used to sequence unknown templates, it can also be used to confirm known sequences, identify single nucleotide polymorphisms, and perform single base extension reactions, amongst others. Cycling of the various steps of the methods leads to detection of additional sequence of the same molecule, one per cycle. When the aim is to sequence a single molecule, or a homogeneous population of molecules, the steps can be carried out in a sequential manner in a flow through or a stop-flow system. In such a flow through or stop flow system, the ternary complex of polymerase-template-nucleotide can be immobilized on beads, and the beads can be localized, for example in a well or within a portion of a microchannel.

Alternatively, some methods can also be adapted to perform massively parallel reactions, to sequence multiple templates at the same time. The massively parallel sequencing can be performed, for example, on isolated regions of a surface, or using beads.

One bead based pyrosequencing platform is recently developed by 454 Life Sciences Corp. Margulies et al., (2005) Nature 437:376-80, incorporated by reference in its entirety. Microbeads are deposited in tiny, picoliter sized wells of a fibre-optic slide, each well can only accommodate a single bead. Each bead carries multiple nucleic acid templates of the same kind (i.e. same sequence), amplified by emulsion PCR. In their system, each bead is surrounded by even smaller beads carrying enzymes required for the pyrophosphate detection. A flow-through system is developed that allows efficient reagent flow and simultaneous extension reactions on each of the template-carrying beads. The light generated by luciferase reaction is detected as well as the position of the well, such that a correlation provides the sequence readout for a particular DNA on a bead in a well at a fixed position. The methods and engineered enzymes described herein can be utilized in such massively parallel systems.

Some embodiments include methods of pyrosequencing wherein the error rate when sequencing a homopolymeric region having multiple, for example, 4 ‘A’ nucleotides or more is lower than when dATPαS is used.

### Apparatus for Obtaining Sequence Information

Some embodiments of the present invention include apparatuses for obtaining sequence information. Such embodiments can include a modified luciferase described herein in fluid communication with a target nucleic acid and polymerase. One embodiment includes a chamber comprising a target nucleic acid in fluid communication with a polymerase and a modified luciferase. By “chamber” is meant any structure that permits co-localization of the site of incorporation of a dNTP into a polynucleotide complementary to the target nucleic acid with luciferase. Although the luciferase and the dNTP incorporation site do not need to be in close proximity, the luciferase should be near enough to the site to permit diffusion or transport of molecules from the site to the area of the luciferase. An example of a “chamber” is a flow cell. However, a “chamber” is not necessarily an enclosed, or even partially enclosed, structure. In some such embodiments, an apparatus can further include a detector associated with the chamber. The detector can detect light emitted by the light emitting activity of the modified luciferase. In some embodiments, the chamber comprises a flow cell. Some apparatuses that can be used with the modified luciferases described herein are described in U.S. Patent Application No. 2006/0040297, incorporated herein by reference in its entirety.

### Methods of Making Luciferases

Some embodiments include methods for producing an engineered luciferase enzyme including the step of performing site directed mutagenesis to produce an enzyme wherein the selectivity of the engineered luciferase toward ATP over dATP is higher than the selectivity of a luciferase enzyme without the modifications.

Methods for performing site-directed mutagenesis are well known to those skilled in the art. Site directed mutagenesis can be performed by overlapping PCR. Site directed mutagenesis can be performed using the QuikChange® mutagenesis kit (Stratagene) or the Transformer Site-Directed Mutagenesis Kit (Clontech). The site directed mutagenesis can be by cassette mutagenesis, where a plasmid is cleaved with one or more restriction enzymes and an oligonucleotide with the mutation of interest is subsequently ligated into the plasmid.

Luciferase can be engineered by expressing it from a heterologous DNA molecule. The luciferase can be encoded from a vector or plasmid. Luciferase can be encoded from a heterologous promoter. The promoter for expressing the luciferase can be an inducible promoter. The engineered luciferase can be expressed in bacteria, including, for example, E. coli. The engineered luciferase can be expressed in a mammalian cell line. The engineered luciferase can be expressed in yeast; for example, Saccharomyces cerevisiae. The luciferase can be engineered to have an epitope tag to facilitate purification. Luciferase can be purified by conventional chromatography or affinity chromatography.

The method of producing an engineered luciferase can include changing one or more of residues corresponding to Arg437, Asp422, Arg537, Ile423, Asp436, and Leu530 in Photinus pyralis luciferase. In preferred embodiments, a modified luciferase can include at least one substitution selected from T214, I232, F295, E354, N197, S198, H244, I423, or any combination thereof. The modifications can be made in luciferases from related organisms. These residues can be changed in any of the ways described above.

The method of producing an engineered luciferase can include mutations including one or more, two or more, or at least the mutations corresponding to Ile423Leu, Asp436Gly, and Leu530Arg in Photinus pyralis luciferase. In preferred embodiments, however, a modified luciferase can include at least one substitution selected from T214A, I232A, F295L, E354K, N197F, S198T, H244F, I423Y, or any combination thereof.

An example of a nucleic acid encoding Photinus pyralis luciferase polypeptide that can be modified to make some of the modified luciferases described herein includes (SEQ ID NO:1):

| | |
|---|---|
| 1 | atggaaaaca tggaaaacga tgaaaatatt gtagttggac ctaaaccgtt ttaccctatc |
| 61 | gaagagggat ctgctggaac acaattacgc aaatacatgg agcgatatgc aaaacttggc |
| 121 | gcaattgctt ttacaaatgc agttactggt gttgattatt cttacgccga atacttggag |
| 181 | aaatcatgtt gtctaggaaa agctttgcaa aattatggtt tggttgttga tggcagaatt |
| 241 | gcgttatgca gtgaaaactg tgaagaattt tttattcctg taatagccgg actgtttata |
| 301 | ggtgtaggtg ttgcacccac taatgagatt tacactttac gtgaactggt tcacagttta |
| 361 | ggtatctcta aaccaacaat tgtatttagt tctaaaaaag gcttagataa agttataaca |
| 421 | gtacagaaaa cagtaactac tattaaaacc attgttatac tagatagcaa agttgattat |
| 481 | cgaggatatc aatgtctgga cacctttata aaaagaaaca ctccaccagg ttttcaagca |
| 541 | tccagtttca aaactgtgga agttgaccgt aaagaacaag ttgctcttat aatgaactct |
| 601 | tcgggttcta ccggtttgcc aaaaggcgta caacttactc acgaaaatac agtcactaga |
| 661 | ttttctcatg ctagagatcc gatttatggt aaccaagttt caccaggcac cgct9tttta |
| 721 | actgtcgttc cattccatca tggttttggt atgttcacta ctctagggta tttaatttgt |
| 781 | ggttttcgtg ttgtaatgtt aacaaaattc gatgaagaaa catttttaaa aactctacaa |
| 841 | gattataaat gtacaagtgt tattcttgta ccgaccttgt ttgcaattct caacaaaagt |
| 901 | gaattactca ataaatacga tttgtcaaat ttagttgaga ttgcatctgg cggagcacct |
| 961 | ttatcaaaag aagttggtga agct9ttgct agacgcttta atcttcccgg tgttcgtcaa |
| 1021 | ggttatggtt taacagaaac aacatctgcc attattatta caccagaagg agacgataaa |
| 1081 | ccaggagctt ctggaaaagt cgtgccgttg tttaaagcaa aagttattga tcttgatacc |
| 1141 | aaaaaatctt taggtcctaa cagacgtgga gaagtttgtg ttaaaggacc tatgcttatg |
| 1201 | aaaggttatg taaataatcc agaagcaaca aaagaactta ttgacgaaga aggttggctg |
| 1261 | cacaccggag atattggata ttatgatgaa gaaaaacatt tctttattgt cgatcgtttg |
| 1321 | aagtctttaa tcaaatacaa aggataccaa gtaccacctg ccgaattaga atccgttctt |
| 1381 | ttgcaacatc catctatctt tgatgctggt gttgccggcg ttcctgatcc tgtagctggc |
| 1441 | gagcttccag gagccgttgt tgtactggaa agcggaaaaa atatgaccga aaaagaagta |
| 1501 | atggattatg ttgcaagtca agtttcaaat gcaaaacgtt tacgtggtgg tgttcgtttt |
| 1561 | gtggatgaag tacctaaagg tcttactgga aaaattgacg gcagagcaat tagagaaatc |
| 1621 | cttaagaaac cagttgctaa gatg |

Accession number: E02267; GI: 2170504

### EXAMPLES

### Example 1

### The Effect of dATP and dATPαS on Luciferase Reaction

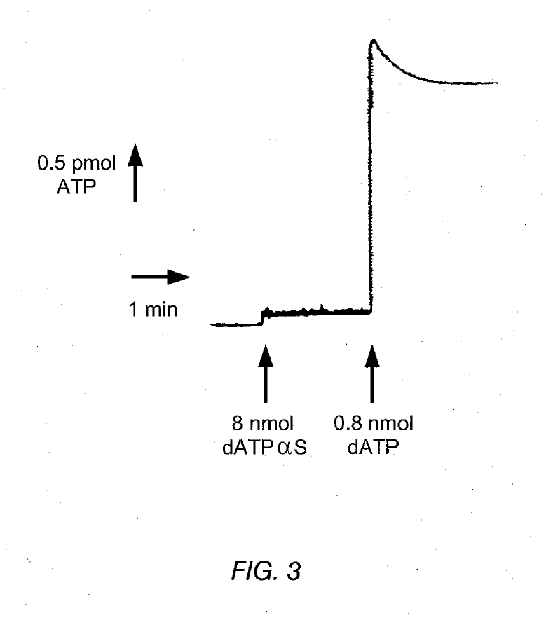

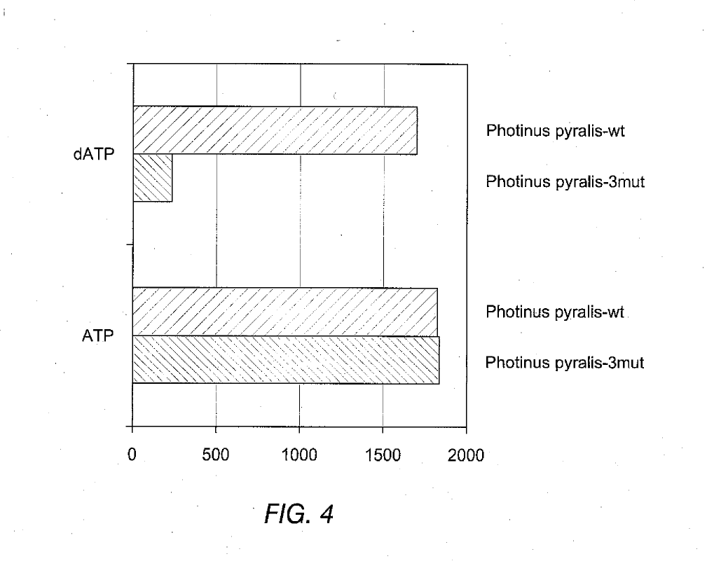

A reaction was started by the addition of 0.1 nmol ATP and 8 nmol ATPαS and the luminescence output was measured (FIG. 3).

### Example 2

### Comparison of the Enzymatic Activity of Wild-Type and Mutated Photinus pyralis Luciferase Using dATP or ATP as Substrates

The mutated luciferase had the Ile423Leu, Asp436Gly and Leu530Arg mutations. 50 nl of ATP (0.1 mM) was added to 10 μl of luciferase solution and the relative light intensity was recorded (duplicate experiments were performed). dATP was added at a 100 mM concentration. The data were adjusted for 60 times more light collected for ATP vs dATP and the concentration.

### Example 3

### Effects of Ile423Leu, Asp436Gly and Leu530Arg mutations in Photinus pyralis luciferase

The Ile423Leu, Asp436Gly and Leu530Arg mutations enhanced the selectivity of luciferase for ATP by five fold. It was observed that the Ile423Leu mutant lowered the K_{M} value for ATP relative to wild type enzyme by 3.6 fold (from 160 μM to 45 μM). An even more dramatic decrease (−20 times improvement) was observed with the Asp436Gly mutant (K_{M}=9 μM).

### Example 4

### Site-Directed Mutagenesis

Site-directed mutagenesis was performed for the wild-type luciferase gene in the pET-28a vector using the Transformer Site-Directed Mutagenesis Kit (Clontech) according to the manufacturer's instructions. The following primer was used to produce the Ile423Leu mutant luciferase: 5′-ggctacattctggagacttagcttactgggacg-3′ (SEQ ID NO:2). Mutated primers were designed according to the types and positions of the substituted amino acids. Different luciferases were assayed using pyrosequencing chemistry and devices.

### Example 5

### Solid-Phase Pyrosequencing

FIG. 5 is a schematic representation of the progress of the enzyme reaction in solid-phase pyrosequencing. The four different nucleotides are added stepwise to the immobilized primed DNA template and the incorporation event is followed using the enzyme ATP sulfurylase and luciferase. After each nucleotide addition, a washing step is performed to allow iterative addition.

### Example 6

### Liquid-Phase Pyrosequencing

FIG. 6 is a schematic representation of the progress of the enzyme reaction in liquid-phase pyrosequencing. Primed DNA template and four enzymes involved in liquid-phase pyrosequencing are placed in a well of a microtiter plate. The four different nucleotides are added stepwise and incorporation is followed using the enzyme ATP sulfurylase and luciferase. The nucleotides are continuously degraded by nucleotide-degrading enzyme allowing addition of subsequent nucleotide. dXTP indicates one of the four nucleotides.

### Example 7

### Pyrosequencing with Engineered Luciferase

A pyrosequencing reaction is performed using dATP, dCTP, dTTP, and dGTP and no dATPαS and substantially purified Photinus pyralis luciferase with the Ile423Leu, Asp436Gly and Leu530Arg mutations. A DNA with a homopolymeric stretch of ‘A’ is sequenced. The results of the sequencing reaction are compared to results using wild-type luciferase and dATPαS instead of dATP.

### Example 8

### Chemical Modification of Luciferase

A lysine group on luciferase is modified with “citraconic anhydride” as described by Dixon et al. (1968) Biochem J. 109:312-4.

### Example 9

### Modified Luciferases

Modified luciferases were constructed from Photinus pyralis luciferase (SEQ ID NO: 3) in the pET41a expression vector. Modified luciferases contained an N-terminal polypeptide for biotin carboxyl carrier protein (BCCP) (SEQ ID NO:4), a Gly-Ser dipeptide linker, and a C-terminal polypeptide for a Photinus pyralis luciferase with thermostability mutations (T214A, 1232A, F295L, and E354K). The position for a substituted residue in a luciferase polypeptide is cited as the position in SEQ ID NO:3.

Photinus pyralis luciferase (SEQ ID NO: 3):
MEDAKNIKKGPAPFYPLEDGTAGEQLHKAMKRYALVPGTIAFTDA
HIEVNITYAEYFEMSVRLAEAMKRYGLNTNHRIVVCSENSLQFFMPVLGA
LFIGVAVAPANDIYNERELLNSMNISQPTVVFVSKKGLQKILNVQKKLPI
IQKIIIMDSKTDYQGFQSMYTFVTSHLPPGFNEYDFVPESFDRDKTIALI
MNSSGSTGLPKGVALPHRTACVRFSHARDPIFGNQIIPDTAILSVVPFHH
GFGMFTTLGYLICGFRVVLMYRFEEELFLRSLQDYKIQSALLVPTLFSFF
AKSTLIDKYDLSNLHEIASGGAPLSKEVGEAVAKRFHLPGIRQGYGLTET
TSAILITPEGDDKPGAVGKVVPFFEAKVVDLDTGKTLGVNQRGELCVRGP
MIMSGYVNNPEATNALIDKDGWLHSGDIAYWDEDEHFFIVDRLKSLIKYK
GYQVAPAELESILLQHPNIFDAGVAGLPDDDAGELPAAVVVLEHGKTMTE
KEIVDYVASQVTTAKKLRGGVVFVDEVPKGLTGKLDARKIREILIKAKKG
GKSKL

Biotin carboxyl carrier protein (BCCP)
(SEQ ID NO: 4):
MEAPAAAEISGHIVRSPMVGTFYRTPSPDAKAFIEVGQKVNVGDTL
CIVEAMKMMNQIEADKSGTVKAILVESGQPVEFDEPLVVIE

Table 1 summarizes modifications present in modified luciferases L1-L23, SIGMA Photinus pyralis luciferase (SIGMA, recombinant luciferase), and ULTRAGLOW luciferase (Promega).

TABLE 1

| | Luciferase | BCCP-fusion | Thermostability |
|---|---|---|---|
| Luciferase | Mutation | polypeptide | mutations |
| L1 | — | + | + |
| L2 | I423L | + | + |
| L3 | Y340F | + | + |
| L4 | D422E | + | + |
| L5 | R437E | + | + |
| L6 | Y340D | + | + |
| L7 | R437D | + | + |
| L8 | L530I | + | + |
| L9 | L530R | + | + |
| L10 | G316A | + | + |
| L11 | N197F | + | + |
| L12 | S198T | + | + |
| L13 | H244F | + | + |
| L14 | I423Y | + | + |
| L15 | D436A | + | + |
| L16 | D436S | + | + |
| L17 | L438Y | + | + |
| L18 | S420T | + | + |
| L19 | T345S | + | + |
| L20 | A317S | + | + |
| L21 | N197C | + | + |
| L22 | K529A | + | + |
| L23 | I423L, D436G, | + | + |
| | L530R | | |
| SIGMA | — | − | − |
| ULTRAGLO | — | − | ND |

Thermostability mutations: T214A, I232A, F295L, and E354K;
+: Present;
−: Absent;
ND: not determined.

### Example 10

### Luciferase Activity Assay

The light emitting activity of various luciferases was measured in the presence of ATP in 200 μl with 1.65 nM luciferase, 300 μM D-luciferin, 25 mM Tricine (pH 7.75), 5 mM Mg(OAc)₂, and 0.1% Tween. Readings of Relative Light Units (RLU) were taken in a Berthold luminometer using the kinetic mode for 90 s. Measurements were recorded for peak RLU values, and for integrated RLU measurements taken every 0.9 s over 90. The amounts of L4, L5, L7, and L22 were not standardized because activity of these luciferases was low. In some assays, 0.023 μg of luciferase may be used. Table 2 shows integrated and peak RLU values for modified luciferases (L1-L23), SIGMA luciferase (Photinus pyralis) (SIGMA, recombinant luciferase), and ULTRAGLOW luciferase (PROMEGA), in the presence of 0.05 μM ATP.

TABLE 2

| | | Activity (0.05 μM ATP) | | | |
|---|---|---|---|---|---|
| Luciferase | Mutation | Integrated (RLU) | Integrated (%) | Peak (RLU) | Peak (%) |
| SIGMA | — | 2055753.00 | 100.00 | 22515.5 | 100.00 |
| L1 | — | 1788519.00 | 87.00 | 19101.50 | 84.84 |
| L2 | I423L | 3570735.50 | 173.69 | 39183.00 | 174.03 |
| L3 | Y340F | 1197725.00 | 58.26 | 12588.00 | 55.91 |
| L4* | D422E | 121226.00 | 5.90 | 1275.00 | 5.66 |
| L5* | R437E | 24908.00 | 1.21 | 259.00 | 1.15 |
| L6 | Y340D | 4109030.00 | 199.88 | 45792.00 | 203.38 |
| L7* | R437D | 2731.50 | 0.13 | 35.50 | 0.16 |
| L8 | L530I | 1530869.50 | 74.47 | 15989.50 | 71.02 |
| L9 | L530R | 4267875.00 | 207.61 | 46870.00 | 208.17 |
| L10 | G316A | 417452.00 | 20.31 | 4382.00 | 19.46 |
| L11 | N197F | 266071.50 | 12.94 | 2786.00 | 12.37 |
| L12 | S198T | 2396028.50 | 116.55 | 27231.00 | 120.94 |
| L13 | H244F | 1275245.50 | 62.03 | 14196.00 | 63.05 |
| L14 | I423Y | 671388.50 | 32.66 | 7139.50 | 31.71 |
| L15 | D436A | 707173.50 | 34.40 | 8121.30 | 36.07 |
| L16 | D436S | 840816.50 | 40.90 | 9484.50 | 42.12 |
| L17 | L438Y | 3896987.50 | 189.56 | 42531.50 | 188.90 |
| L18 | S420T | 1873415.50 | 91.13 | 20217.00 | 89.79 |
| L19 | T345S | 3949003.00 | 192.10 | 42754.50 | 189.89 |
| L20 | A317S | 868953.50 | 42.27 | 9159.50 | 40.68 |
| L21 | N197C | 36033.00 | 1.75 | 463.00 | 2.06 |
| L22* | K529A | 3377941.00 | 164.32 | 35071.50 | 155.77 |
| L23 | I423L, D436G, | 2348574.00 | 114.24 | 26373.00 | 117.13 |
| | L530R | | | | |
| ULTRAGLO | — | 15107331.5 | 734.88 | 563394.5 | 2502.25 |

*Luciferase protein undiluted in assay;
+: Present;
−: Absent.

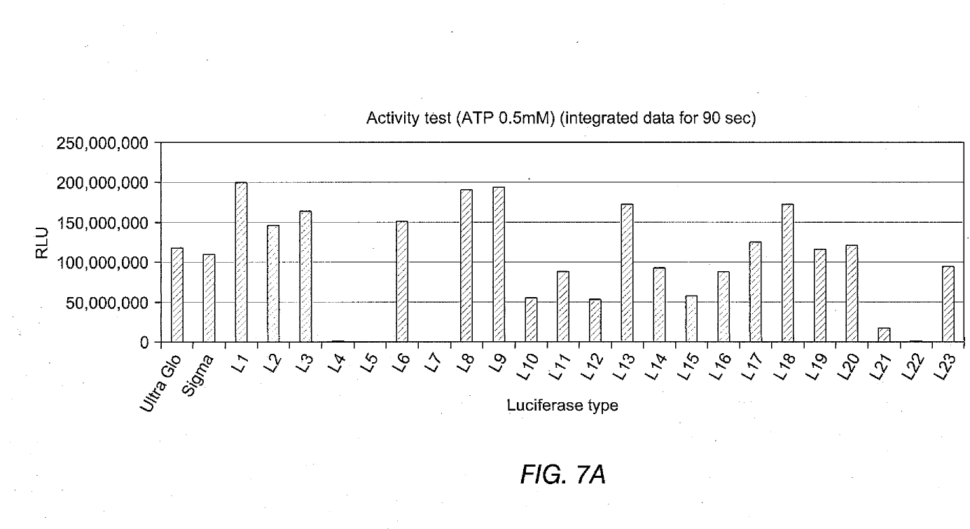

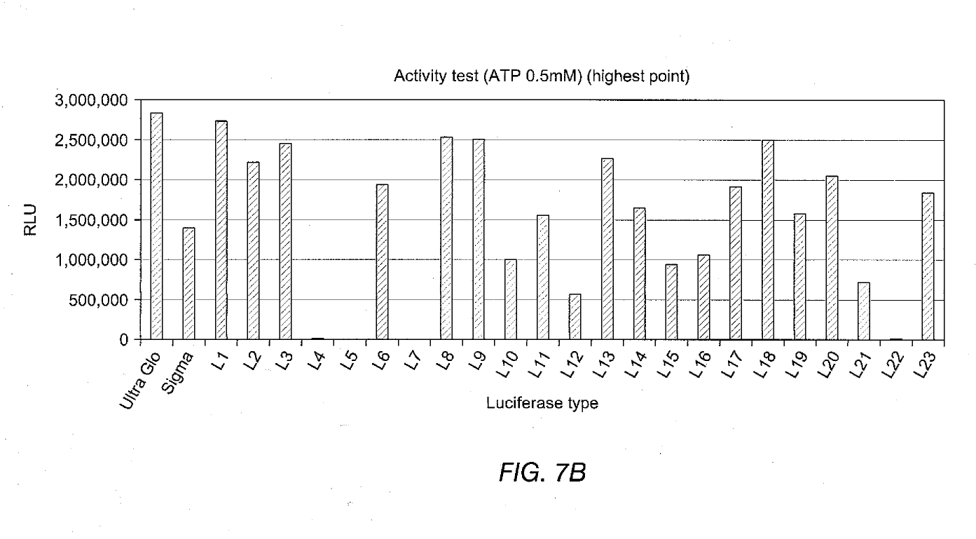

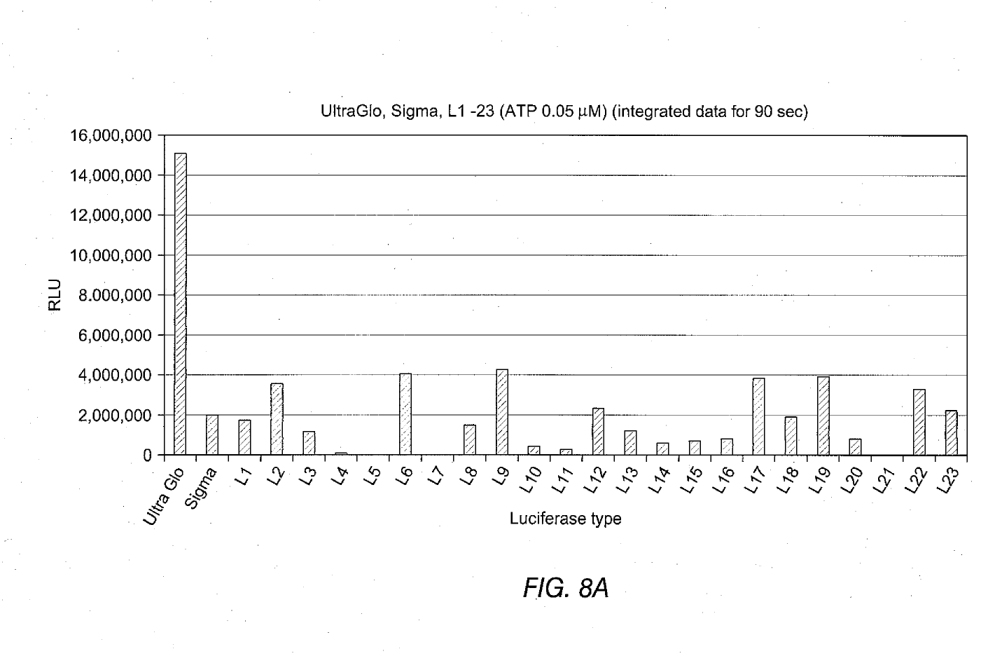

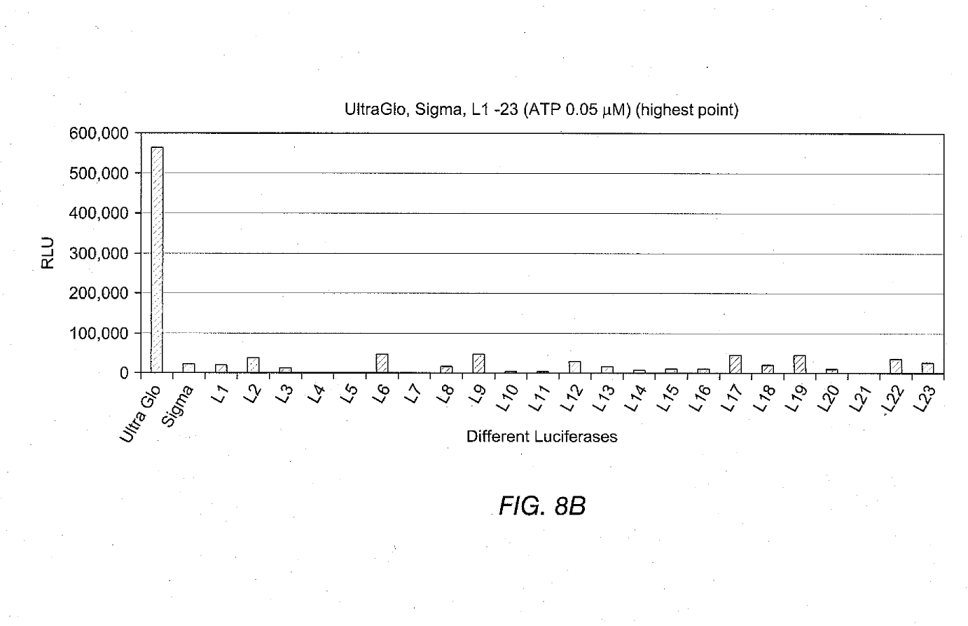

FIGS. 7A and 7B show integrated and peak RLU measurements, respectively, for modified luciferases (L1-L23), SIGMA luciferase (Photinus pyralis), and ULTRAGLOW luciferase (PROMEGA) in the presence of 0.5 mM ATP. FIGS. 8A and 8B show integrated and peak RLU measurements, respectively, for modified luciferases (L1-L23), SIGMA luciferase (Photinus pyralis), and ULTRAGLOW luciferase (PROMEGA) in the presence of 0.05 μM ATP.

### Example 11

### ATP/dATP Selectivity Assay

The selectivity of ATP over dATP for various luciferases was measured by determining the ratio of luciferase activity in the presence of ATP compared with the luciferase activity in the presence of dATP. Activities were measured for integrated RLU values as described above. In one set of measurements, activity ratios were determined from luciferase activities measured in the presence of 0.05 μM ATP or 0.5 μM dATP.

In another set of measurements, activity ratios were averaged from a series of ratios determined from luciferase activities for the various concentrations of ATP or dATP shown in Table 3.

TABLE 3

| ATP (μM) | dATP (μM) |
|---|---|
| 0.05 | 0.5 |
| 0.1 | 1.0 |
| 0.2 | 2.0 |
| 1.0 | 10.0 |
| 5.0 | 50 |
| 10.0 | 100 |

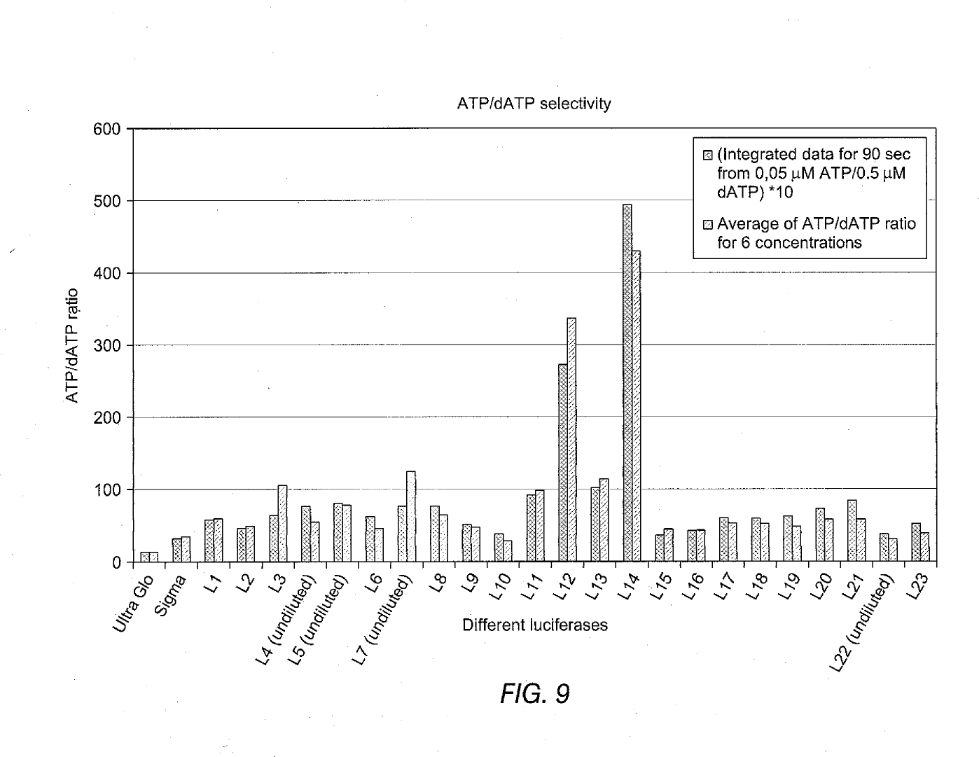

Table 4 shows activity ratios for modified luciferases (L1-L23), SIGMA luciferase (Photinus pyralis), and ULTRAGLOW luciferase (PROMEGA) in the presence of 0.05 μM ATP or 0.5 μM ATP, and in the presence of the concentrations of ATP or dATP shown in Table 3. FIG. 9 shows the data of Table 4 in a graph.

TABLE 4

| | | Activity ratio | |
|---|---|---|---|
| | | 0.05 μM ATP/ | ATP/dATP |
| Luciferase | Mutation | 0.5 μM dATP | (Average) |
| SIGMA | — | 32.70 | 35.12 |
| L1 | — | 58.80 | 60.08 |
| L2 | I423L | 46.30 | 49.90 |
| L3 | Y340F | 64.10 | 104.38 |
| L4 * | D422E | 76.50 | 54.71 |
| L5 * | R437E | 80.90 | 78.17 |
| L6 | Y340D | 62.10 | 45.80 |
| L7 * | R437D | 77.40 | 124.24 |
| L8 | L530I | 77.60 | 65.12 |
| L9 | L530R | 50.90 | 47.57 |
| L10 | G316A | 36.80 | 28.10 |
| L11 | N197F | 90.50 | 97.18 |
| L12 | S198T | 272.10 | 335.00 |
| L13 | H244F | 100.40 | 112.70 |
| L14 | I423Y | 493.50 | 429.02 |
| L15 | D436A | 35.50 | 44.56 |
| L16 | D436S | 41.80 | 43.93 |
| L17 | L438Y | 60.60 | 53.33 |
| L18 | S420T | 59.20 | 52.33 |
| L19 | T345S | 61.50 | 48.49 |
| L20 | A317S | 73.80 | 58.83 |
| L21 | N197C | 83.30 | 58.82 |
| L22 * | K529A | 37.50 | 31.02 |
| L23 | I423L, D436G, | 52.60 | 39.08 |
| | L530R | | |
| ULTRAGLO | — | 13.20 | 13.42 |

* Luciferase protein undiluted in assay;
+: Present;
−: Absent.

### Example 12

### Activity of modified luciferase associated with a substrate

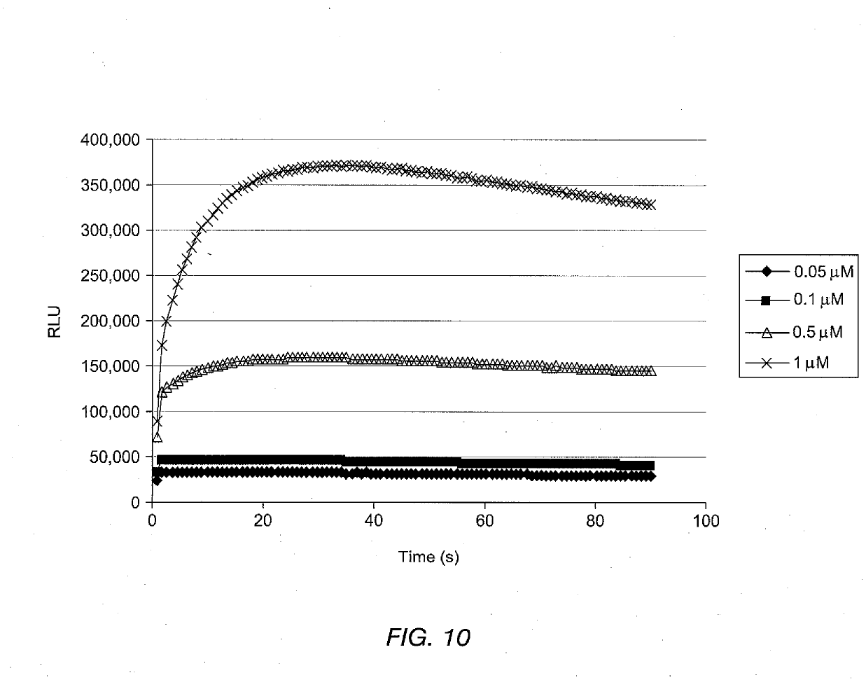

Luciferase L19 was immobilized on M280 beads (Invitrogen). The activity of the luciferase attached to the 3.2 μl beads (1.61×10⁸ beads/ml) was measured in the presence of 0.05 μM, 0.1 μM, 0.5 μM, 1.0 μM ATP according to the light emitting assay described above. FIG. 10 shows the RLU of the L19-beads over time.

### Example 13

### Apparatus for Obtaining Sequence Information

A flow cell contains a fluid medium containing a target nucleic acid associated with a substrate. The target nucleic acid is contacted with a polymerase. dATP, dCTP, dGTP, and dTTP are sequentially added to the fluid medium of the flow cell and contact the target nucleic acid and polymerase. On extension of a polynucleotide complementary to the target nucleic acid, PPi is released. An ATP sulfurylase and modified luciferase are associated with a bead, the bead is in fluid communication with the target nucleic acid and polymerase. The fluid volume contains adenyl sulfate and luciferin. On PPi release, the ATP sulfurylase combines PPi with adenyl sulfate to form ATP. ATP and luciferin bind to the modified luciferase. Light is emitted from the modified luciferase indicating the incorporation of one or more dNTPs into the polynucleotide complementary to the target sequence. A detector couple to the flow cell detects the light emitted by the modified luciferase and incorporation of a specific nucleotide is detected.

### Example 14

### Chemical Modification of Luciferase

SIGMA luciferase was modified with acetic anhydride and citraconic anhydride to modify Lys residues. The activity of the luciferase was tested as described above. The modified luciferase retained its activity. SIGMA luciferase was modified with Diethyl pyrocarbonate (DEPC) to modify His residues. The activity of the luciferase was tested as described above. The modified luciferase retained its activity.

### Example 15

### Effect of pH on Luciferase Selectivity

The ATP/dATP selectivity of SIGMA luciferase was tested as described above, using conditions at pH 7.75, 7.2, 6.8, or 6.55. The ATP/dATP selectivity of the luciferase increased in experiments where a lower pH was tested.

While preferred embodiments of the present invention have been shown and described herein, it will be obvious to those skilled in the art that such embodiments are provided by way of example only. Numerous variations, changes, and substitutions will occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

What is claimed is:

1. A modified luciferase comprising a selectivity for ATP over dATP that is at least three-fold greater than the selectivity for ATP over dATP of an unmodified luciferase from the same organism.

2. The modified luciferase of claim 1, wherein the selectivity for ATP over dATP is at least eight-fold greater then the selectivity for ATP over dATP of an unmodified luciferase from the same organism.

3. The modified luciferase of claim 1, further comprising a light emitting activity that is greater than the light emitting activity of said unmodified luciferase.

4. The modified luciferase of claim 1 having an altered amino acid sequence as compared to the amino acid sequence of an unmodified luciferase from the same organism.

5. The modified luciferase of claim 4, wherein said modified luciferase is from Photinus pyralis.

6. The modified luciferase of claim 5, wherein the altered amino acid sequence comprises an amino acid substitution.

7. The modified luciferase of claim 6, wherein the amino acid substitution is located in the primary amino acid sequence between the N-terminus and the first amino acid forming the active site of said modified luciferase.

8. The modified luciferase of claim 6, wherein the amino acid substitution comprises a conservative amino acid substitution.

9. The modified luciferase of claim 6, wherein the amino acid substitution is selected from the group consisting of N197, S198, H244, I423 and any combination thereof.

10. The modified luciferase of claim 9, wherein the amino acid substitution is selected from the group consisting of N197F, S198T, H244F, I423Y and any combination thereof.

11. The modified luciferase of claim 1, further comprising greater thermostability than said unmodified luciferase.

12. The modified luciferase of claim 6, further comprising greater thermostability than said unmodified luciferase.

13. The modified luciferase of claim 12, wherein the amino acid substitution is selected from the group consisting of T214, I232, F295, E354, and any combination thereof.

14. The modified luciferase of claim 13, wherein the amino acid substitution is selected from the group consisting of T214A, I232A, F295L, E354K, and any combination thereof.

15. The modified luciferase of claim 1 further comprising a biotin binding moiety.

16. The modified luciferase of claim 6, further comprising a biotin binding moiety.

17. The modified luciferase of claim 16, wherein the biotin binding moiety comprises a polypeptide.

18. The modified luciferase of claim 17, wherein the polypeptide has at least 70% identity to SEQ ID NO:1.

19. The modified luciferase of claim 18, wherein the amino acid substitution is selected from the group consisting of T214A, I232A, F295L, E354K, N197F, S198T, H244F, I423Y and any combination thereof.

20. The modified luciferase of claim 6, wherein the altered amino acid sequence comprises an amino acid substitution within 10 Angstroms of a binding site for a nucleotide or a binding site for luciferin in the tertiary structure of the modified luciferase.

21. The modified luciferase of claim 20, wherein the amino acid substitution is within 5 Angstroms of the binding site for a nucleotide or the binding site for luciferin.

22. The modified luciferase of claim 1, associated with a solid support.

23. The modified luciferase of claim 21, wherein the solid support comprises a particle.


SEQUENCE LISTING (16 sequences)

SEQ ID NO: 1 - DNA, 1644 bp, Photinus pyralis
atggaaaacatggaaaacgatgaaaatattgtagttggacctaaaccgttttaccctatcgaagagggatctgctggaacacaattacgcaaatacatggagcgatatgcaaaacttggcgcaattgcttttacaaatgcagttactggtgttgattattcttacgccgaatacttggagaaatcatgttgtctaggaaaagctttgcaaaattatggtttggttgttgatggcagaattgcgttatgcagtgaaaactgtgaagaattttttattcctgtaatagccggactgtttataggtgtaggtgttgcacccactaatgagatttacactttacgtgaactggttcacagtttaggtatctctaaaccaacaattgtatttagttctaaaaaaggcttagataaagttataacagtacagaaaacagtaactactattaaaaccattgttatactagatagcaaagttgattatcgaggatatcaatgtctggacacctttataaaaagaaacactccaccaggttttcaagcatccagtttcaaaactgtggaagttgaccgtaaagaacaagttgctcttataatgaactcttcgggttctaccggtttgccaaaaggcgtacaacttactcacgaaaatacagtcactagattttctcatgctagagatccgatttatggtaaccaagtttcaccaggcaccgctgttttaactgtcgttccattccatcatggttttggtatgttcactactctagggtatttaatttgtggttttcgtgttgtaatgttaacaaaattcgatgaagaaacatttttaaaaactctacaagattataaatgtacaagtgttattcttgtaccgaccttgtttgcaattctcaacaaaagtgaattactcaataaatacgatttgtcaaatttagttgagattgcatctggcggagcacctttatcaaaagaagttggtgaagctgttgctagacgctttaatcttcccggtgttcgtcaaggttatggtttaacagaaacaacatctgccattattattacaccagaaggagacgataaaccaggagcttctggaaaagtcgtgccgttgtttaaagcaaaagttattgatcttgataccaaaaaatctttaggtcctaacagacgtggagaagtttgtgttaaaggacctatgcttatgaaaggttatgtaaataatccagaagcaacaaaagaacttattgacgaagaaggttggctgcacaccggagatattggatattatgatgaagaaaaacatttctttattgtcgatcgtttgaagtctttaatcaaatacaaaggataccaagtaccacctgccgaattagaatccgttcttttgcaacatccatctatctttgatgctggtgttgccggcgttcctgatcctgtagctggcgagcttccaggagccgttgttgtactggaaagcggaaaaaatatgaccgaaaaagaagtaatggattatgttgcaagtcaagtttcaaatgcaaaacgtttacgtggtggtgttcgttttgtggatgaagtacctaaaggtcttactggaaaaattgacggcagagcaattagagaaatccttaagaaaccagttgctaagatg

SEQ ID NO: 2 - DNA, 33 bp, Artificial Sequence
  synthetic oligonucleotide
ggctacattctggagacttagcttactgggacg

SEQ ID NO: 3 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 4 - protein, 87 aa, Escherichia coli
MEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 5 - protein, 548 aa, Luciola cruciata [sequence omitted]

SEQ ID NO: 6 - protein, 548 aa, Luciola lateralis
MENMENDENIVYGPEPFYPIEEGSAGAQLRKYMDRYAKLGAIAFTNALTGVDYTYAEYLEKSCCLGEALKNYGLVVDGRIALCSENCEEFFIPVLAGLFIGVGVAPTNEIYTLRELVHSLGISKPTIVFSSKKGLDKVITVQKTVATIKTIVILDSKVDYRGYQSMDNFIKKNTPQGFKGSSFKTVEVNRKEQVALIMNSSGSTGLPKGVQLTHENAVTRFSHARDPIYGNQVSPGTAILTVVPFHHGFGMFTTLGYLTCGFRIVMLTKFDEETFLKTLQDYKCSSVILVPTLFAILNRSELLDKYDLSNLVEIASGGAPLSKEIGEAVARRFNLPGVRQGYGLTETTSAIIITPEGDDKPGASGKVVPLFKAKVIDLDTKKTLGPNRRGEVCVKGPMLMKGYVDNPEATREIIDEEGWLHTGDIGYYDEEKHFFIVDRLKSLIKYKGYQVPPAELESVLLQHPNIFDAGVAGVPDPIAGELPGAVVVLEKGKSMTEKEVMDYVASQVSNAKRLRGGVRFVDEVPKGLTGKIDGKAIREILKKPVAKM

SEQ ID NO: 7 - protein, 548 aa, Luciola mingrelica
MEMEKEENVVYGPLPFYPIEEGSAGIQLHKYMHQYAKLGAIAFSNALTGVDISYQEYFDITCRLAEAMKNFGMKPEEHIALCSENCEEFFIPVLAGLYIGVAVAPTNEIYTLRELNHSLGIAQPTIVFSSRKGLPKVLEVQKTVTCIKKIVILDSKVNFGGHDCMETFIKKHVELGFQPSSFVPIDVKNRKQHVALLMNSSGSTGLPKGVRITHEGAVTRFSHAKDPIYGNQVSPGTAILTVVPFHHGFGMFTTLGYFACGYRVVMLTKFDEELFLRTLQDYKCTSVILVPTLFAILNKSELIDKFDLSNLTEIASGGAPLAKEVGEAVARRFNLPGVRQGYGLTETTSAFIITPEGDDKPGASGKVVPLFKVKVIDLDTKKTLGVNRRGEICVKGPSLMLGYSNNPEATRETIDEEGWLHTGDIGYYDEDEHFFIVDRLKSLIKYKGYQVPPAELESVLLQHPNIFDAGVAGVPDPDAGELPGAVVVMEKGKTMTEKEIVDYVNSQVVNHKRLRGGVRFVDEVPKGLTGKIDAKVIREILKKPQAKM

SEQ ID NO: 8 - protein, 548 aa, pyrocoelia miyako
MEDDSKHIMHGHRHSILWEDGTAGEQLHKAMKRYAQVPGTIAFTDAHAEVNITYSEYFEMSCRLAETMKRYGLGLQHHIAVCSETSLQFFMPVCGALFIGVGVAPTNDIYNERELYNSLFISQPTIVFCSKRALQKILGVQKKLPVIQKIVILDSREDYMGKQSMYSFIESHLPAGFNEYDYIPDSFDRETATALIMNSSGSTGLPKGVDLTHMNVCVRFSHCRDPVFGNQIIPDTAILTVIPFHHVFQMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAIIITPEGDDKPGACGKVVPFFTAKIVDLDTGKTLGVNQRGELCVKGPMIMKGYVNNPEATNALIDKDGWLHSGDIAYYDKDGHFFIVDRLKSLIKYKGYQVPPAELESILLQHPFIFDAGVAGIPDPDAGELPAAVVVLEEGKMMTEQEVMDYVAGQVTASKRLRGGVKFVDEVPKGLTGKIDSRKIREILTMGQKSKL

SEQ ID NO: 9 - protein, 550 aa, Photinus pyralis; identical to SEQ ID NO: 3

SEQ ID NO: 10 - protein, 547 aa, Lampyris noctiluca
MEDAKNIMHGPAPFYPLEDGTAGEQLHKAMKRYAQVPGTIAFTDAHAEVNITYSEYFEMACRLAETMKRYGLGLQHHIAVCSENSLQFFMPVCGALFIGVGVASTNDIYNERELYNSLSISQPTIVSCSKRALQKILGVQKKLPIIQKIVILDSREDYMGKQSMYSFIESHLPAGFNEYDYIPDSFDRETATALIMNSSGSTGLPKGVELTHQNVCVRFSHCRDPVFGNQIIPDTAILTVIPFHHGFGMFTTLGYLTCGFRIVLMYRFEEELFLRSLQDYKIQSALLVPTLFSFFAKSTLVDKYDLSNLHEIASGGAPLAKEVGEAVAKRFKLPGIRQGYGLTETTSAIIITPEGDDKPGACGKVVPFFSAKIVDLDTGKTLGVNQRGELCVKGPMIMKGYVNNPEATSALIDKDGWLHSGDIAYYDKDGHFFIVDRLKSLIKYKGYQVPPAELESILLQHPFIFDAGVAGIPDPDAGELPAAVVVLEEGKTMTEQEVMDYVAGQVTASKRLRGGVKFVDEVPKGLTGKIDGRKIREILMMGKKSKL

SEQ ID NO: 11 - protein, 554 aa, Photuris pennsylvanica
MSIENNILIGPPPYYPLEEGTAGEQLHRAISRYAAVPGTLAYTDVHTELEVTYKEFLDVTCRLAEAMKNYGLGLGIHTISVCSENCVQFFMPICAALYVGVATAPTNDIYNERELYNSLSISGIPTVVFTSRNSLQKILGVQSRLPIIKKIIILDGKKDYLGYQSMQSFMKEHVPANFNVSAFKPLSFDLDRVACIMNSSGSTGLPKGVPISHRNTIYRFSHCRDPVFGNQIIPDTTILCAVPFHHAFGTFTNLGYLICGFHVVLMYRFNEHLFLQTLQDYKCQSALLVPTVLAFLAKNPLVDKYDLSNLHEIASGGAPLSKEISEIAAKRFKLPGIRQGYGLTETTCAIVITAEGEFKLGAVGKVVPFYSLKVLDLNTGKKLGPNERGEICFKGPMIMKGYINNPEATRELIDEEGWIHSGDIGYFDEDGHVYIVDRLKSLIKYKGYQVPPAELEALLLQHPFIEDAGVAGVPDEVAGDLPGAVVVLKEGKSITEKEIQDYVAGQVTSSKKLRGGVEFVKEVPKGFTGKIDTRKIKEILIKAQKGKSKSKAKL

SEQ ID NO: 12 - protein, 548 aa, Phengodes sp.
MIKMEEEHVMPGAMPRDLLFEGTAGQQLHRALYKHSYFPEAIVDSHTHEIISYAKILDMSCRLAVSFQKYGLTQNNIIGICSENNLNFFNPVIAAFYLGITVATVNDTYTDRELSETLNITKPQMLFCSKGISLPIVMKTMKIMPYVQKLLIIDSMQDIGGIECVHSFVSRYTDEHFDPLKFVPLDFDPREQVALIMTSSGTTGLPKGVMLTHRNICVRFVHSRDPLFGTRFIPETSILSLVPFHHAFGMFTTLSYFIVGLKIVMMKRFDGELFLKTIQNYKIPTIVIAPPVMVFLAKSHLVDKYDLSSIKEIATGGAPLGPALANAVAKRLKLGGIIQGYGLTETCCAVLITPHNKIKTGSTGGIVLPYVTAKIVDTKTGKNLGPNQTGELCFKSDIIMKGYYQNEEETRLVIDKDGWLHSGDIGYYDTDGNFHIVDRLKELIKYKAYQVAPAELEALLLQHPYIADAGVTGIPDEEAGELPAACVVLEPGKTMTEKEVMDYIAERVTPTKRLRGGVLFVNNIPKGATGKLVRTELRRLLTQRAAKL

SEQ ID NO: 13 - protein, 543 aa, Pyrophorus plagiophthalamus
MMKREKNVVYGPEPLHPLEDLTAGEMLFRALRKHSHLPQALVDVYGEEWISYKEFFETTCLLAQSLHNCGYKMSDVVSICAENNKRFFVPIIAAWYIGMIVAPVNEGYIPDELCKVMGISRPQLVFCTKNILNKVLEVQSRTDFIKRIIILDAVENIHGCESLPNFISRYSDGNIANFKPLHYDPVEQVAAILCSSGTTGLPKGVMQTHRNVCVRLIHALDPRVGTQLIPGVTVLVYLPFFHAFGFSINLGYFMVGLRVIMLRRFDQEAFLKAIQDYEVRSVINVPAIILFLSKSPLVDKYDLSSLRELCCGAAPLAKEVAEIAVKRLNLPGIRCGFGLTESTSANIHSLRDEFKSGSLGRVTPLMAAKIADRETGKALGPNQVGELCIKGPMVSKGYVNNVEATKEAIDDDGWLHSGDFGYYDEDEHFYVVDRYKELIKYKGSQVAPAELEEILLKNPCIRDVAVVGIPDLEAGELPSAFVVIQPGKEITAKEVYDYLAERVSHTKYLRGGVRFVDSIPRNVTGKITRKELLKQLLEKSSKL

SEQ ID NO: 14 - protein, 543 aa, Pyrophorus plagiophthalamus
MMKAEKNVIYGPEPLHPLEDLTAGEMLFRALRKHSHLPQALVDVFGDESLSYKEFFEATCLLAQSLHNCGYKMNDVVSICAENNKRFFIPIIAAWYIGMIVAPVNESYIPDELCKVMGISKPQIVFCTKNILNKVLEVQSRTNFIKRIIILDTVENIHGCESLPNFISRYSDGNIANFKPLHYDPVEQVAAILCSSGTTGLPKGVMQTHQNICVRLIHALDPRAGTQLIPGVTVLVYLPFFHAFGFSINLGYFMVGLRVIMLRRFDQEAFLKAIQDYEVRSVINVPAIILFLSKSPLVDKYDLSSLRELCCGAAPLAKEVAEVAVKRLNLPGIRCGFGLTESTSANIHSLGDEFKSGSLGRVTPLMAAKIADRETGKALGPNQVGELCVKGPMVSKGYVNNVEATKEAIDDDGWLHSGDFGYYDEDEHFYVVDRYKELIKYKGSQVAPAELEEILLKNPCIRDVAVVGIPDLEAGELPSAFVVKQPGKEITAKEVYDYLAERVSHTKYLRGGVRFVDSIPRNVTGKITRKELLKQLLEKSSKL

SEQ ID NO: 15 - protein, 545 aa, Photuris pennsylvanica
MEDKNILYGPEPFYPLADGTAGEQMFYALSRYADISGCIALTNAHTKENVLYEEFLKLSCRLAESFKKYGLKQNDTIAVCSENGLQFFLPLIASLYLGIIAAPVSDKYIERELIHSLGIVKPRIIFCSKNTFQKVLNVKSKLKYVETIIILDLNEDLGGYQCLNNFISQNSDINLDVKKFKPNSFNRDDQVALVMFSSGTTGVSKGVMLTHKNIVARFSHCKDPTFGNAINPTTAILTVIPFHHGFGMTTTLGYFTCGFRVALMHTFEEKLFLQSLQDYKVESTLLVPTLMAFFAKSALVEKYDLSHLKEIASGGAPLSKEIGEMVKKRFKLNFVRQGYGLTETTSAVLITPDTDVRPGSTGKIVPFHAVKVVDPTTGKILGPNETGELYFKGDMIMKSYYNNEEATKAIINKDGWLRSGDIAYYDNDGHFYIVDRLKSLIKYKGYQVAPAEIEGILLQHPYIVDAGVTGIPDEAAGELPAAGVVVQTGKYLNEQIVQNFVSSQVSTAKWLRGGVKFLDEIPKGSTGKIDRKVLRQMFEKHKSKL

SEQ ID NO: 16 - DNA, 20 bp, Artificial Sequence
  synthetic oligonucleotide
accttgagtaccatctagga